(19) United States
(12) Patent Application Publication
(10) Pub. No.: US 2012/0214848 A1
(43) Pub. Date: Aug. 23, 2012

(54) [1,2,4]OXADIAZOL-3-YL ACID SALTS AND CRYSTALLINE FORMS AND THEIR PREPARATION

(75) Inventors: Geoff G. Zhang, Vernon Hills, IL (US); Paul J. Brackemeyer, Mount Prospect, IL (US); Shaung Chen, Gurnee, IL (US); Thomas D. Gordon, Medway, MA (US)

(73) Assignee: ABBOTT LABORATORIES, Abbott Park, IL (US)

(21) Appl. No.: 13/399,088

(22) Filed: Feb. 17, 2012

Related U.S. Application Data
(60) Provisional application No. 61/444,351, filed on Feb. 18, 2011.

(51) Int. Cl. A61K 31/4439; A61P 35/00; A61K 31/4245; A61P 37/06; C07D 413/04; C07D 271/06

(52) U.S. Cl. 514/340; 546/269.4; 548/131; 514/364

(57) ABSTRACT

The present invention relates to salts and crystalline forms of [1,2,4]oxadiazol-3-yl]-phenoxy}-cycloalkyl carboxylic acids, processes for their preparation, pharmaceutical compositions comprising such compounds, and methods of using them.

### CROSS REFERENCE TO RELATED APPLICATION

This application claims priority to U.S. Provisional Application Ser. No. 61/444,351 filed on Feb. 18, 2011, the contents of which are incorporated herein.

### BACKGROUND OF THE INVENTION

The invention relates to certain compounds having therapeutic utility and to processes for their preparation. More particularly, the present invention relates to salts, solvates and crystalline forms of [1,2,4]oxadiazol-3-yl]-phenoxy}-cycloalkyl carboxylic acids, more particularly 3-{3-chloro-4-[5-(5-chloro-6-isopropoxy-pyridin-3-yl)-[1,2,4]oxadiazol-3-yl]-phenoxy}-cyclobutane carboxylic acid, 3-(2-(4-(3-(3-chloro-4-isopropoxyphenyl)-1,2,4-oxadiazol-5-yl)phenyl)propan-2-ylamino)propanoic acid and (1R,3S)-3-(4-(5-(5-chloro-6-isopropoxypyridin-3-yl)-1,2,4-oxadiazol-3-yl)phenylamino)cyclopentane carboxylic acid which demonstrate activity as sphingosine-1-phosphate-1 (S1P₁) receptor agonists.

Sphingosine-1-phosphate (S1P) is part of the sphingomyelin biosynthetic pathway and is known to affect multiple biological processes. S1P is formed through phosphorylation of sphingosine by sphingosine kinases (SK1 and SK2) and it is degraded through cleavage by sphingosine lyase to form palmitaldehyde and phosphoethanolamine or through dephosphorylation by phospholipid phosphatases. It is present at high levels (˜500 nM) in serum, and it is found in most tissues. It can be synthesized in a wide variety of cells in response to several stimuli, which include cytokines, growth factors and G protein-coupled receptor (GPCR) ligands. The GPCRs that bind SP (currently known as the S1P receptors S1P1-5), couple through pertusis toxin sensitive (Gi) pathways as well as pertusis toxin insensitive pathways to stimulate a variety of processes. The individual receptors of the S1P family are both tissue and response specific and so are attractive as therapeutic targets.

S1P evokes many responses from cells and tissues. In particular, S1P has been shown to be an agonist at all five GPCRs, S1P1 (Edg-1), S1P2 (Edg-5), S1P3 (Edg-3), S1P4 (Edg-6) and S1P5 (Edg-8). The action of SP at the SP receptors has been linked to resistance to apoptosis, changes in cellular morphology, cell migration, growth, differentiation, cell division, angiogenesis and modulation of the immune system via alterations of lymphocyte trafficking. Therefore, S1P receptors are targets for therapy of, for example, neoplastic diseases, autoimmune disorders and tissue rejection in transplantation. These receptors also share 50-55% amino acid identity with three other lysophospholipid receptors, LPA1, LPA2, and LPA3 of the structurally related lysophosphatidic acid (LPA).

GPCRs are excellent drug targets with numerous examples of marketed drugs across multiple disease areas. GPCRs are cell surface receptors that bind hormones on the extracellular surface of the cell and transduce a signal across the cellular membrane to the inside of the cell. The internal signal is amplified through interaction with G proteins which in turn interact with various second messenger pathways. This transduction pathway is manifested in downstream cellular responses that include cytoskeletal changes, cell motility, proliferation, apoptosis, secretion and regulation of protein expression, to name a few. SIP receptors make good drug targets because individual receptors are expressed in different tissues and signal through different pathways, making the individual receptors both tissue and response specific. Tissue specificity of the SIP receptors is desirable because development of an agonist or antagonist selective for one receptor localizes the cellular response to tissues containing that receptor, limiting unwanted side effects. Response specificity of the SIP receptors is also of importance because it allows for the development of agonists or antagonists that initiate or suppress certain cellular responses without affecting other responses. For example, the response specificity of the SIP receptors could allow for an S1P mimetic that initiates platelet aggregation without affecting cell morphology.

The physiologic implications of stimulating individual SIP receptors are largely unknown due in part to a lack of receptor type selective ligands. Isolation and characterization of SIP analogs that have potent agonist or antagonist activity for S1P receptors have been limited.

S1P1 for example is widely expressed, and the knockout causes embryonic lethality due to large vessel rupture. Adoptive cell transfer experiments using lymphocytes from SIP1 knockout mice have shown that S1P1 deficient lymphocytes sequester to secondary lymph organs. Conversely, T cells overexpressing S1P1 partition preferentially into the blood compartment rather than secondary lymph organs. These experiments provide evidence that S1P1 is the main sphingosine receptor involved in lymphocyte homing and trafficking to secondary lymphoid compartments.

### BRIEF DESCRIPTION OF THE DRAWINGS

FIG. 1 is the powder X-ray diffractogram of 3-{3-chloro-4-[5-(5-chloro-6-isopropoxypyridin-3-yl)-[1,2,4]oxadiazol-3-yl]-phenoxy}-cyclobutanecarboxylic acid anhydrate and peak listing.

FIG. 1A is the crystallographic information of 3-{3-chloro-4-[5-(5-chloro-6-isopropoxypyridin-3-yl)-[1,2,4]oxadiazol-3-yl]-phenoxy}-cyclobutane carboxylic acid anhydrate.

FIG. 2 is the powder X-ray diffractogram of 3-{3-chloro-4-[5-(5-chloro-6-isopropoxypyridin-3-yl)-[1,2,4]oxadiazol-3-yl]-phenoxy}-cyclobutanecarboxylic acid TRIS salt and peak listing.

FIG. 3 is the powder X-ray diffractogram of 3-{3-chloro-4-[5-(5-chloro-6-isopropoxypyridin-3-yl)-[1,2,4]oxadiazol-3-yl]-phenoxy}-cyclobutane carboxylic acid sodium salt hydrate and peak listing.

FIG. 3A is the crystallographic information of 3-{3-chloro-4-[5-(5-chloro-6-isopropoxypyridin-3-yl)-[1,2,4]oxadiazol-3-yl]-phenoxy}-cyclobutane carboxylic acid sodium salt hydrate.

FIG. 4 is the powder X-ray diffractogram of 3-{3-chloro-4-[5-(5-chloro-6-isopropoxypyridin-3-yl)-[1,2,4]oxadiazol-3-yl]-phenoxy}-cyclobutane carboxylic acid potassium salt and peak listing.

FIG. 5 is the powder X-ray diffractogram of 3-{3-chloro-4-[5-(5-chloro-6-isopropoxypyridin-3-yl)-[1,2,4]oxadiazol-3-yl]-phenoxy}-cyclobutanecarboxylic acid magnesium salt and peak listing.

FIG. 6 is the powder X-ray diffractogram of 3-{3-chloro-4-[5-(5-chloro-6-isopropoxypyridin-3-yl)-[1,2,4]oxadiazol-3-yl]-phenoxy}-cyclobutanecarboxylic acid meglumine salt and peak listing.

FIG. 7 is the powder X-ray diffractogram of 3-(2-(4-(3-(3-chloro-4-isopropoxyphenyl)-1,2,4-oxadiazol-5-yl)phenyl)propan-2-ylamino)propanoic acid methanol solvate and peak listing.

FIG. 8 is the powder X-ray diffractogram of 3-(2-(4-(3-(3-chloro-4-isopropoxyphenyl)-1,2,4-oxadiazol-5-yl)phenyl)propan-2-ylamino)propanoic acid ethanol solvate and peak listing.

FIG. 9 is the powder X-ray diffractogram of 3-(2-(4-(3-(3-chloro-4-isopropoxyphenyl)-1,2,4-oxadiazol-5-yl)phenyl)propan-2-ylamino)propanoic acid 2-propanol solvate and peak listing.

FIG. 10 is the powder X-ray diffractogram of 3-(2-(4-(3-(3-chloro-4-isopropoxyphenyl)-1,2,4-oxadiazol-5-yl)phenyl)propan-2-ylamino)propanoic acid 1-propanol solvate and peak listing.

FIG. 11 is the powder X-ray diffractogram of 3-(2-(4-(3-(3-chloro-4-isopropoxyphenyl)-1,2,4-oxadiazol-5-yl)phenyl)propan-2-ylamino)propanoic acid ethyl acetate solvate and peak listing.

FIG. 12 is the powder X-ray diffractogram of 3-(2-(4-(3-(3-chloro-4-isopropoxyphenyl)-1,2,4-oxadiazol-5-yl)phenyl)propan-2-ylamino)propanoic acid toluene solvate and peak listing.

FIG. 13 is the powder X-ray diffractogram of 3-(2-(4-(3-(3-chloro-4-isopropoxyphenyl)-1,2,4-oxadiazol-5-yl)phenyl)propan-2-ylamino)propanoic acid hydrochloride salt and peak listing.

FIG. 14 is the powder X-ray diffractogram of 3-(2-(4-(3-(3-chloro-4-isopropoxyphenyl)-1,2,4-oxadiazol-5-yl)phenyl)propan-2-ylamino)propanoic acid bitartrate salt and peak listing.

FIG. 15 is the powder X-ray diffractogram of 3-(2-(4-(3-(3-chloro-4-isopropoxyphenyl)-1,2,4-oxadiazol-5-yl)phenyl)propan-2-ylamino)propanoic acid bimalate salt and peak listing.

FIG. 16 is the powder X-ray diffractogram of (1R,3S)-3-(4-(5-(5-chloro-6-isopropoxypyridin-3-yl)-1,2,4-oxadiazol-3-yl)phenylamino)cyclopentanecarboxylic acid methanol solvate and peak listing.

FIG. 17 is the powder X-ray diffractogram of (1R,3S)-3-(4-(5-(5-chloro-6-isopropoxypyridin-3-yl)-1,2,4-oxadiazol-3-yl)phenylamino)cyclopentanecarboxylic acid ethanol solvate and peak listing.

FIG. 18 is the powder X-ray diffractogram of (1R,3S)-3-(4-(5-(5-chloro-6-isopropoxypyridin-3-yl)-1,2,4-oxadiazol-3-yl)phenylamino)cyclopentanecarboxylic acid 2-propanol solvate and peak listing.

FIG. 19 is the powder X-ray diffractogram of (1R,3S)-3-(4-(5-(5-chloro-6-isopropoxypyridin-3-yl)-1,2,4-oxadiazol-3-yl)phenylamino)cyclopentanecarboxylic acid 1-propanol solvate and peak listing.

FIG. 20 is the powder X-ray diffractogram of (1R,3S)-3-(4-(5-(5-chloro-6-isopropoxypyridin-3-yl)-1,2,4-oxadiazol-3-yl)phenylamino)cyclopentanecarboxylic acid sodium salt and peak listing.

FIG. 21 is the powder X-ray diffractogram of (1R,3S)-3-(4-(5-(5-chloro-6-isopropoxypyridin-3-yl)-1,2,4-oxadiazol-3-yl)phenylamino)cyclopentanecarboxylic acid potassium salt and peak listing.

FIG. 22 is the powder X-ray diffractogram of (1R,3S)-3-(4-(5-(5-chloro-6-isopropoxypyridin-3-yl)-1,2,4-oxadiazol-3-yl)phenylamino)cyclopentanecarboxylic acid nicotinamide co-crystal and peak listing.

### SUMMARY OF THE INVENTION

In one aspect the present invention relates to salts and crystalline forms of 3-{3-chloro-4-[5-(5-chloro-6-isopropoxy-pyridin-3-yl)-[1,2,4]oxadiazol-3-yl]-phenoxy}-cyclobutanecarboxylic acid, and more particularly, isolated forms of these salts and crystals. In another aspect the present invention relates to salts, solvates and crystalline forms of 3-(2-(4-(3-(3-chloro-4-isopropoxyphenyl)-1,2,4-oxadiazol-5-yl)phenyl)propan-2-ylamino)propanoic acid. In yet another aspect the present invention relates to salts, solvates and crystalline forms of (1R,3S)-3-(4-(5-(5-chloro-6-isopropoxypyridin-3-yl)-1,2,4-oxadiazol-3-yl)phenylamino)cyclopentane carboxylic acid. The salts and crystalline forms obtained can be substantially pure and demonstrate characteristic peaks as shown in their X-ray powder diffraction pattern. Processes for preparing such salts and crystalline forms are also contemplated. In particular, the invention is directed to crystalline forms, TRIS salts, sodium salts, potassium salts, meglumine salts, magnesium salts, methanol solvates, ethanol solvates, 1-propanol solvates, 2-propanol solvates, ethyl acetate solvates, toluene solvates, hydrochloride salts, bitartrate salts and nicotinamide co-crystals.

Another aspect of the invention relates to a method of treating disorders mediated by sphingosine-1-phosphates comprising the step of administering a therapeutically effective amount of a salt or crystalline form of 3-{3-chloro-4-[5-(5-chloro-6-isopropoxypyridin-3-yl)-[1,2,4]oxadiazol-3-yl]-phenoxy}-cyclobutanecarboxylic acid, a composition comprising the same, or a mixture containing the compound or composition, to a host mammal, particularly a human, in need of such treatment. More particularly, the method relates to administering TRIS, sodium, potassium, magnesium and meglumine salts of 3-{3-chloro-4-[5-(5-chloro-6-isopropoxy-pyridin-3-yl)-[1,2,4]oxadiazol-3-yl]-phenoxy}-cyclobutanecarboxylic acid.

Another aspect of the invention relates to a method of treating disorders mediated by sphingosine-1-phosphates comprising the step of administering a therapeutically effective amount of a salt or crystalline form of 3-(2-(4-(3-(3-chloro-4-isopropoxyphenyl)-1,2,4-oxadiazol-5-yl)phenyl)propan-2-ylamino)propanoic acid, a composition comprising the same, or a mixture containing the compound or composition, to a host mammal, particularly a human, in need of such treatment. More particularly, the method relates to administering methanol solvate, ethanol solvate, 2-propanol solvate, 1-propanol solvate, ethyl acetate solvate, toluene solvate, hydrochloride salt, bitartrate salt, and bimalate salt of 3-(2-(4-(3-(3-chloro-4-isopropoxyphenyl)-1,2,4-oxadiazol-5-yl)phenyl)propan-2-ylamino)propanoic acid.

Another aspect of the invention relates to a method of treating disorders mediated by sphingosine-1-phosphates comprising the step of administering a therapeutically effective amount of a salt or crystalline form of (1R,3S)-3-(4-(5-(5-chloro-6-isopropoxypyridin-3-yl)-1,2,4-oxadiazol-3-yl)phenylamino)cyclopentane carboxylic acid, a composition comprising the same, or a mixture containing the compound or composition, to a host mammal, particularly a human, in need of such treatment. More particularly, the method relates to administering methanol solvate, ethanol solvate, 2-propanol solvate, 1-propanol solvate, sodium salt, nicotinamide co-crystal or potassium salt of (1R,3S)-3-(4-(5-(5-chloro-6-isopropoxypyridin-3-yl)-1,2,4-oxadiazol-3-yl)phenylamino)cyclopentane carboxylic acid.

The compounds, compositions comprising the compounds, methods for making the compounds and methods for treating and preventing conditions and disorders by administering the compounds are further described herein.

In a first embodiment the invention provides a compound having the systematic name 3-{3-chloro-4-[5-(5-chloro-6-isopropoxy-pyridin-3-yl)-[1,2,4]oxadiazol-3-yl]phenoxy}-cyclobutane carboxylic acid (Compound 1) in a salt or crystalline form.

In a second embodiment the invention provides a compound according to the first embodiment, wherein the crystalline form is free acid anhydrate, characterized by a powder X-ray diffraction pattern having at least one peak selected from those at 6.4, 7.6, 9.8, 14.1, 14.7, 16.7, 17.4, 18.5, 19.2, 19.7, 20.7, 21.3, 22.7, 24.3, 24.9, 26.1 or 26.5, each peak being ±0.2 degrees 2θ, when measured at about 25° C. with Cu K_{α} radiation at 1.5418 Å.

In a third embodiment the invention provides a compound according to the first embodiment, wherein the salt form is TRIS salt, characterized by a powder X-ray diffraction pattern having at least one peak selected from those at 3.4, 6.8, 10.2, 11.7, 13.4, 15.7, 18.3, 18.9, 21.6, 21.8, 22.2 or 25.0, each peak being ±0.2 degrees 2θ, when measured at about 25° C. with Cu K_{α} radiation at 1.5418 Å.

In a fourth embodiment the invention provides a compound according to the first embodiment, wherein the crystalline form is sodium salt hydrate, characterized by a powder X-ray diffraction pattern having at least one peak selected from those at 3.2, 6.4, 9.6, 12.7, 16.0, 16.6, 17.0, 19.2, 22.5, 24.3, 25.2 or 26.5, each peak being ±0.2 degrees 2θ, when measured at about 25° C. with Cu K_{α} radiation at 1.5418 Å.

In a fifth embodiment the invention provides a compound according to the first embodiment, wherein the salt form is potassium salt, characterized by a powder X-ray diffraction pattern having at least one peak selected from those at 4.2, 6.3, 10.6, 16.0, 16.2, 19.0, 19.4, 19.6, 20.3 or 23.3, each peak being ±0.2 degrees 2θ, when measured at about 25° C. with Cu K_{α} radiation at 1.5418 Å.

In a sixth embodiment the invention provides a compound according to the first embodiment, wherein the crystalline form is magnesium salt, characterized by a powder X-ray diffraction pattern having at least one peak selected from those at 3.5, 7.0, 10.6, 11.5, 12.3, 13.3, 14.1, 17.0, 17.7 or 18.4, each peak being ±0.2 degrees 2θ, when measured at about 25° C. with Cu K_{α} radiation at 1.5418 Å.

In a seventh embodiment the invention provides a compound according to the first embodiment, wherein the crystalline form is meglumine salt, characterized by a powder X-ray diffraction pattern having at least one peak selected from those at 3.1, 6.1, 9.0, 11.2, 12.7, 14.7, 15.2, 17.0, 17.8, 18.0, 19.5, 20.3 or 20.7, each peak being ±0.2 degrees 2θ, when measured at about 25° C. with Cu K_{α} radiation at 1.5418 Å.

In an eighth embodiment the invention provides the free acid anhydrate according to the first embodiment wherein the lattice type is triclinic, the space group is P-1, a is about 6.500 Å, b is about 11.814 Å, c is about 13.903 Å, α is about 88.21°, β is about 7.20°, γ is about 78.23°, and Z is two.

In a ninth embodiment the invention provides the sodium salt hydrate according to the first embodiment wherein the lattice type is triclinic, the space group is P-1, a is about 5.490 Å, b is about 8.441 Å, c is about 27.342 Å, α is about 88.84°, β is 88.20°, and γ is about 76.19°, and Z is two.

In a tenth embodiment the invention provides a pharmaceutical composition comprising a compound according to the first embodiment and one or more pharmaceutically acceptable excipients.

In an eleventh embodiment the invention provides a pharmaceutical composition comprising a compound according to the second embodiment and one or more pharmaceutically acceptable excipients.

In a twelfth embodiment the invention provides a pharmaceutical composition comprising a compound according to the third embodiment and one or more pharmaceutically acceptable excipients.

In a thirteenth embodiment the invention provides a pharmaceutical composition comprising a compound according to the fourth embodiment and one or more pharmaceutically acceptable excipients.

In a fourteenth embodiment the invention provides a pharmaceutical composition comprising a compound according to the fifth embodiment and one or more pharmaceutically acceptable excipients.

In a fifteenth embodiment the invention provides a pharmaceutical composition comprising a compound according to the sixth embodiment and one or more pharmaceutically acceptable excipients.

In a sixteenth embodiment the invention provides a pharmaceutical composition comprising a compound according to the seventh embodiment and one or more pharmaceutically acceptable excipients.

In a seventeenth embodiment the invention provides a pharmaceutical composition comprising a compound according to the eighth embodiment and one or more pharmaceutically acceptable excipients.

In an eighteenth embodiment the invention provides a pharmaceutical composition comprising a compound according to the ninth embodiment and one or more pharmaceutically acceptable excipients.

In a nineteenth embodiment the invention provides a process for preparing a pharmaceutical composition of Compound 1 comprising dissolving a compound according to the first embodiment in a pharmaceutically acceptable solvent or mixture of solvents.

In a twentieth embodiment the invention provides a process for preparing a pharmaceutical composition of Compound 1 comprising dissolving a compound according to the second embodiment in a pharmaceutically acceptable solvent or mixture of solvents.

In a twenty-first embodiment the invention provides a process for preparing a pharmaceutical composition of Compound 1 comprising dissolving a compound according to the third embodiment in a pharmaceutically acceptable solvent or mixture of solvents.

In a twenty-second embodiment the invention provides a process for preparing a pharmaceutical composition of Compound 1 comprising dissolving a compound according to the fourth embodiment in a pharmaceutically acceptable solvent or mixture of solvents.

In a twenty-third embodiment the invention provides a process for preparing a pharmaceutical composition of Compound 1 comprising dissolving a compound according to the fifth embodiment in a pharmaceutically acceptable solvent or mixture of solvents.

In a twenty-fourth embodiment the invention provides a process for preparing a pharmaceutical composition of Compound 1 comprising dissolving a compound according to the sixth embodiment in a pharmaceutically acceptable solvent or mixture of solvents.

In a twenty-fifth embodiment the invention provides a process for preparing a pharmaceutical composition of Compound 1 comprising dissolving a compound according to the seventh embodiment in a pharmaceutically acceptable solvent or mixture of solvents.

In a twenty-sixth embodiment the invention provides a process for preparing a pharmaceutical composition of Compound 1 comprising dissolving a compound according to the eighth embodiment in a pharmaceutically acceptable solvent or mixture of solvents.

In a twenty-seventh embodiment the invention provides a process for preparing a pharmaceutical composition of Compound 1 comprising dissolving a compound according to the ninth embodiment in a pharmaceutically acceptable solvent or mixture of solvents.

In a twenty-eighth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the first embodiment or (b) a pharmaceutical composition comprising a compound according to the first embodiment and one or more pharmaceutically acceptable excipients.

In a twenty-ninth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the second embodiment or (b) a pharmaceutical composition comprising a compound according to the second embodiment and one or more pharmaceutically acceptable excipients.

In a thirtieth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the third embodiment or (b) a pharmaceutical composition comprising a compound according to the third embodiment and one or more pharmaceutically acceptable excipients.

In a thirty-first embodiment the invention provides a method of treating a disease characterized by sphingosine-1-phosphate dysfunction and/or overexpression of sphingosine-1-phosphate, comprising administering to a subject having the disease a therapeutically effective amount of (a) a compound of according to the fourth embodiment or (b) a pharmaceutical composition comprising a compound according to the fourth embodiment and one or more pharmaceutically acceptable excipients.

In a thirty-second embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the fifth embodiment or (b) a pharmaceutical composition comprising a compound according to the fifth embodiment and one or more pharmaceutically acceptable excipients.

In a thirty-third embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the sixth embodiment or (b) a pharmaceutical composition comprising a compound according to the sixth embodiment and one or more pharmaceutically acceptable excipients.

In a thirty-fourth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the seventh embodiment or (b) a pharmaceutical composition comprising a compound according to the seventh embodiment and one or more pharmaceutically acceptable excipients.

In a thirty-fifth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the eighth embodiment or (b) a pharmaceutical composition comprising a compound according to the eighth embodiment and one or more pharmaceutically acceptable excipients.

In a thirty-sixth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the ninth embodiment or (b) a pharmaceutical composition comprising a compound according to the ninth embodiment and one or more pharmaceutically acceptable excipients.

In a thirty-seventh embodiment the invention provides a compound having the systematic name 3-(2-(4-(3-(3-chloro-4-isopropoxyphenyl)-1,2,4-oxadiazol-5-yl)phenyl)propan-2-ylamino)propanoic acid (Compound 2) in a salt, solvate or crystalline form.

In a thirty-eighth embodiment the invention provides the compound according to the thirty-eighth embodiment, wherein the crystalline form is methanol solvate, characterized by a powder X-ray diffraction pattern having at least one peak selected from those at 5.9, 6.8, 10.8, 11.9, 12.5, 13.6, 16.6, 17.1, 17.8, 18.6, 21.8, 24.0 or 27.81, each peak being ±0.2 degrees 2θ, when measured at about 25° C. with Cu K_{α} radiation at 1.5418 Å.

In a thirty-ninth embodiment the invention provides the compound according to the thirty-eighth embodiment, wherein the crystalline form is ethanol solvate, characterized by a powder X-ray diffraction pattern having at least one peak selected from those at 5.9, 6.7, 10.7, 12.5, 13.4, 16.5, 16.8, 17.7, 20.1, 21.6, 23.8 or 27.5 each peak being ±0.2 degrees 2θ, when measured at about 25° C. with Cu K_{α} radiation at 1.5418 Å.

In a fortieth embodiment the invention provides the compound according to the thirty-eighth embodiment, wherein the crystalline form is 2-propanol solvate, characterized by a powder X-ray diffraction pattern having at least one peak selected from those at 5.0, 6.0, 9.6, 11.0, 15.5, 17.0, 18.0, 20.8, 21.9, 22.6, 24.1 or 25.4 each peak being ±0.2 degrees 2θ, when measured at about 25° C. with Cu K_{α} radiation at 1.5418 Å.

In a forty-first embodiment the invention provides the compound according to the thirty-eighth embodiment, wherein the crystalline form is 1-propanol solvate, characterized by a powder X-ray diffraction pattern having at least one peak selected from those at 6.9, 8.2, 9.9, 11.0, 11.4, 13.8, 15.0, 16.0, 16.3, 17.2, 18.6, 20.0, 23.1, 23.9, 24.5 or 25.3, each peak being ±0.2 degrees 2θ, when measured at about 25° C. with Cu K_{α} radiation at 1.5418 Å.

In a forty-second embodiment the invention provides the compound according to the thirty-eighth embodiment, wherein the crystalline form is ethyl acetate solvate, characterized by a powder X-ray diffraction pattern having at least one peak selected from those at 3.3, 6.6, 9.8, 11.2, 13.4, 14.5, 15.5, 16.4, 20.3, 23.0 or 24.5, each peak being ±0.2 degrees 2θ, when measured at about 25° C. with Cu K_{α} radiation at 1.5418 Å.

In a forty-third embodiment the invention provides the compound according to the thirty-eighth embodiment, wherein the crystalline form is toluene solvate, characterized by a powder X-ray diffraction pattern having at least one peak selected from those at 3.3, 6.6, 9.9, 13.1, 13.4, 13.7, 15.5, 16.4, 17.3, 20.4, 23.1 or 24.6, each peak being ±0.2 degrees 2θ, when measured at about 25° C. with Cu K_{α} radiation at 1.5418 Å.

In a forty-fourth embodiment the invention provides the compound according to the thirty-eighth embodiment, wherein the salt form is hydrochloride salt, characterized by a powder X-ray diffraction pattern having at least one peak selected from those at 6.1, 9.2, 12.1, 12.9, 15.9, 17.3, 17.5, 17.8, 18.2, 20.5, 22.1, 23.1, 24.4, 24.8, 25.4, 27.5 or 28.2, each peak being ±0.2 degrees 2θ, when measured at about 25° C. with Cu K_{α} radiation at 1.5418 Å.

In a forty-fifth embodiment the invention provides the compound according to the thirty-eighth embodiment, wherein the salt form is bitartrate salt, characterized by a powder X-ray diffraction pattern having at least one peak selected from those at 4.0, 10.1, 11.2, 11.6, 14.1, 14.7, 15.7, 17.1, 18.5, 18.9, 19.6, 23.6, 24.1, 24.4, 25.0, 25.4, 25.7, 26.5 or 27.0, each peak being ±0.2 degrees 2θ, when measured at about 25° C. with Cu K_{α} radiation at 1.5418 Å.

In a forty-sixth embodiment the invention provides the compound according to the thirty-eighth embodiment, wherein the salt form is bimalate salt, characterized by a powder X-ray diffraction pattern having at least one peak selected from those at 10.2, 11.3, 11.8, 14.3, 15.7, 16.6, 18.9, 19.9, 23.6, 24.2, 24.5, 24.9, 26.0 or 26.9, each peak being ±0.2 degrees 2θ, when measured at about 25° C. with Cu K_{α} radiation at 1.5418 Å.

In a forty-seventh embodiment the invention provides a pharmaceutical composition comprising a compound according to the thirty-seventh embodiment and one or more pharmaceutically acceptable excipients.

In a forty-eighth embodiment the invention provides a pharmaceutical composition comprising a compound according to the thirty-eighth embodiment and one or more pharmaceutically acceptable excipients.

In a forty-ninth embodiment the invention provides a pharmaceutical composition comprising a compound according to the thirty-ninth embodiment and one or more pharmaceutically acceptable excipients.

In a fiftieth embodiment the invention provides a pharmaceutical composition comprising a compound according to the fortieth embodiment and one or more pharmaceutically acceptable excipients.

In a fifty-first embodiment the invention provides a pharmaceutical composition comprising a compound according to the forty-first embodiment and one or more pharmaceutically acceptable excipients.

In a fifty-second embodiment the invention provides a pharmaceutical composition comprising a compound according to the forty-second embodiment and one or more pharmaceutically acceptable excipients.

In a fifty-third embodiment the invention provides a pharmaceutical composition comprising a compound according to the forty-third embodiment and one or more pharmaceutically acceptable excipients.

In a fifty-fourth embodiment the invention provides a pharmaceutical composition comprising a compound according to the forty-fourth embodiment and one or more pharmaceutically acceptable excipients.

In a fifty-fifth embodiment the invention provides a pharmaceutical composition comprising a compound according to the forty-fifth embodiment and one or more pharmaceutically acceptable excipients.

In a fifty-sixth embodiment the invention provides a pharmaceutical composition comprising a compound according to the forty-sixth embodiment and one or more pharmaceutically acceptable excipients.

In a fifty-seventh embodiment the invention provides a process for preparing a pharmaceutical composition of Compound 2 comprising dissolving a compound according to the thirty-seventh embodiment in a pharmaceutically acceptable solvent or mixture of solvents.

In a fifty-eighth embodiment the invention provides a process for preparing a pharmaceutical composition of Compound 2 comprising dissolving a compound according to the thirty-eighth embodiment in a pharmaceutically acceptable solvent or mixture of solvents.

In a fifty-ninth embodiment the invention provides a process for preparing a pharmaceutical composition of Compound 2 comprising dissolving a compound according to the thirty-ninth embodiment in a pharmaceutically acceptable solvent or mixture of solvents.

In a sixtieth embodiment the invention provides a process for preparing a pharmaceutical composition of Compound 2 comprising dissolving a compound of according to the fortieth embodiment in a pharmaceutically acceptable solvent or mixture of solvents.

In a sixty-first embodiment the invention provides a process for preparing a pharmaceutical composition of Compound 2 comprising dissolving a compound of according to the forty-first embodiment in a pharmaceutically acceptable solvent or mixture of solvents.

In a sixty-second embodiment the invention provides a process for preparing a pharmaceutical composition of Compound 2 comprising dissolving a compound according to the forty-second embodiment in a pharmaceutically acceptable solvent or mixture of solvents.

In a sixty-third embodiment the invention provides a process for preparing a pharmaceutical composition of Compound 2 comprising dissolving a compound according to the forty-third embodiment in a pharmaceutically acceptable solvent or mixture of solvents.

In a sixty-fourth embodiment the invention provides a process for preparing a pharmaceutical composition of Compound 2 comprising dissolving a compound according to the forty-fourth embodiment in a pharmaceutically acceptable solvent or mixture of solvents.

In a sixty-fifth embodiment the invention provides a process for preparing a pharmaceutical composition of Compound 2 comprising dissolving a compound according to the forty-fifth embodiment in a pharmaceutically acceptable solvent or mixture of solvents.

In a sixty-sixth embodiment the invention provides a process for preparing a pharmaceutical composition of Compound 2 comprising dissolving a compound according to the forty-sixth embodiment in a pharmaceutically acceptable solvent or mixture of solvents.

In a sixty-seventh embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the thirty-seventh embodiment or (b) a pharmaceutical composition comprising a compound according to the thirty-seventh embodiment and one or more pharmaceutically acceptable excipients.

In a sixty-eighth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the thirty-eighth embodiment or (b) a pharmaceutical composition comprising a compound according to the thirty-eighth embodiment and one or more pharmaceutically acceptable excipients.

In a sixty-ninth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the thirty-ninth embodiment or (b) a pharmaceutical composition comprising a compound according to the thirty-ninth embodiment and one or more pharmaceutically acceptable excipients.

In a seventieth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the fortieth embodiment or (b) a pharmaceutical composition comprising a compound according to the fortieth embodiment and one or more pharmaceutically acceptable excipients.

In a seventy-first embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the forty-first embodiment or (b) a pharmaceutical composition comprising a compound according to the forty-first embodiment and one or more pharmaceutically acceptable excipients.

In a seventy-second embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the forty-second embodiment or (b) a pharmaceutical composition comprising a compound according to the forty-second embodiment and one or more pharmaceutically acceptable excipients.

In a seventy-third embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the forty-third embodiment or (b) a pharmaceutical composition comprising a compound according to the forty-third embodiment and one or more pharmaceutically acceptable excipients.

In a seventy-fourth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the forty-fourth embodiment or (b) a pharmaceutical composition comprising a compound according to the forty-fourth embodiment and one or more pharmaceutically acceptable excipients.

In a seventy-fifth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the forty-fifth embodiment or (b) a pharmaceutical composition comprising a compound according to the forty-fifth embodiment and one or more pharmaceutically acceptable excipients.

In a seventy-sixth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the forty-sixth embodiment or (b) a pharmaceutical composition comprising a compound according to the forty-sixth embodiment and one or more pharmaceutically acceptable excipients.

In a seventy-seventh embodiment the invention provides a compound having the systematic name (1R,3S)-3-(4-(5-(5-chloro-6-isopropoxypyridin-3-yl)-1,2,4-oxadiazol-3-yl)phenylamino)cyclopentanecarboxylic acid (Compound 3) in a salt, solvate or crystalline form.

In a seventy-eighth embodiment the invention provides the compound according to the seventy-seventh embodiment, wherein the crystalline form is methanol solvate, characterized by a powder X-ray diffraction pattern having at least one peak selected from those at 4.7, 5.8, 6.6, 7.0, 9.9, 10.3, 13.6, 13.9, 14.3, 14.7, 15.9, 16.4, 17.3, 17.5, or 20.0 each peak being ±0.2 degrees 2θ, when measured at about 25° C. with Cu K_{α} radiation at 1.5418 Å.

In an seventy-ninth embodiment the invention provides the compound according to the seventy-seventh embodiment, wherein the crystalline form is ethanol solvate, characterized by a powder X-ray diffraction pattern having at least one peak selected from those at 6.3, 7.4, 11.6, 12.5, 14.7, 15.6, 17.1, 18.7, 19.1, 20.2, 23.1, 23.3, 24.5 or 25.7 each peak being ±0.2 degrees 2θ, when measured at about 25° C. with Cu K_{α} radiation at 1.5418 Å.

In an eightieth embodiment the invention provides the compound according to the seventy-seventh embodiment, wherein the crystalline form is 2-propanol solvate, characterized by a powder X-ray diffraction pattern having at least one peak selected from those at 5.6, 6.9, 9.3, 13.8, 15.4, 16.8, 18.7, 19.7, 22.0, 22.8 or 25.3 each peak being ±0.2 degrees 2θ, when measured at about 25° C. with Cu K_{α} radiation at 1.5418 Å.

In an eighty-first embodiment the invention provides the compound according to the seventy-seventh embodiment, wherein the crystalline form is 1-propanol solvate, characterized by a powder X-ray diffraction pattern having at least one peak selected from those at 5.6, 6.8, 8.8, 11.6, 13.1, 13.6, 15.1, 17.6, 18.1 or 20.4 each peak being ±0.2 degrees 2θ, when measured at about 25° C. with Cu K_{α} radiation at 1.5418 Å.

In an eighty-second embodiment the invention provides the compound according to the seventy-seventh embodiment, wherein the salt form is sodium salt, characterized by a powder X-ray diffraction pattern having at least one peak selected from those at 2.8, 5.6, 8.4, 11.2, 14.0, 15.3, 16.8, 18.1, 18.7, 19.8, 20.5, 24.9 or 26.5 each peak being ±0.2 degrees 2θ, when measured at about 25° C. with Cu K_{α} radiation at 1.5418 Å.

In an eighty-third embodiment the invention provides the compound according to the seventy-seventh embodiment, wherein the salt form is potassium salt, characterized by a powder X-ray diffraction pattern having at least one peak selected from those at 4.5, 4.7, 8.7, 10.1, 10.9, 12.4, 12.9, 13.5, 13.9, 14.1, 16.1, 16.7, 17.1, 17.5, 17.9, 21.9, 23.5, 24.0 or 24.5 each peak being ±0.2 degrees 2θ, when measured at about 25° C. with Cu K_{α} radiation at 1.5418 Å.

In an eighty-fourth embodiment the invention provides a pharmaceutical composition comprising a compound according to the seventy-seventh embodiment wherein the salt form is nicotinamide co-crystal, characterized by a powder X-ray diffraction pattern having at least one peak selected from those at 6.4, 7.3, 11.4, 11.8, 14.8, 17.5, 22.3, 25.4, 25.9, or 27.3 each peak being ±0.2 degrees 2θ, when measured at about 25° C. with Cu K_{α} radiation at 1.5418 Å.

In an eighty-fifth embodiment the invention provides a pharmaceutical composition comprising a compound according to the seventy-seventh embodiment, and one or more pharmaceutically acceptable excipients.

In an eighty-sixth embodiment the invention provides a pharmaceutical composition comprising a compound according to the seventy-eighth embodiment and one or more pharmaceutically acceptable excipients.

In an eighty-seventh embodiment the invention provides a pharmaceutical composition comprising a compound according to the seventy-ninth embodiment and one or more pharmaceutically acceptable excipients.

In an eighty-eighth embodiment the invention provides a pharmaceutical composition comprising a compound according to the eightieth embodiment and one or more pharmaceutically acceptable excipients.

In an eighty-ninth embodiment the invention provides a pharmaceutical composition comprising a compound according to the eighty-first embodiment and one or more pharmaceutically acceptable excipients.

In a ninetieth embodiment the invention provides a pharmaceutical composition comprising a compound according to the eighty-second embodiment and one or more pharmaceutically acceptable excipients.

In a ninety-first embodiment the invention provides a pharmaceutical composition comprising a compound according to the eighty-third embodiment and one or more pharmaceutically acceptable excipients.

In a ninety-second embodiment the invention provides a pharmaceutical composition comprising a compound according to the eighty-fourth embodiment and one or more pharmaceutically acceptable excipients.

In a ninety-third embodiment the invention provides a process for preparing a pharmaceutical composition of Compound 3 comprising dissolving a compound according to the seventy-seventh embodiment in a pharmaceutically acceptable solvent or mixture of solvents.

In a ninety-fourth embodiment the invention provides a process for preparing a pharmaceutical composition of Compound 3 comprising dissolving a compound according to the seventy-eighth embodiment in a pharmaceutically acceptable solvent or mixture of solvents.

In a ninety-fifth embodiment the invention provides a process for preparing a pharmaceutical composition of Compound 3 comprising dissolving a compound according to the seventy-ninth embodiment in a pharmaceutically acceptable solvent or mixture of solvents.

In a ninety-sixth embodiment the invention provides a process for preparing a pharmaceutical composition of Compound 3 comprising dissolving a compound of according to the eightieth embodiment in a pharmaceutically acceptable solvent or mixture of solvents.

In a ninety-seventh embodiment the invention provides a process for preparing a pharmaceutical composition of Compound 3 comprising dissolving a compound according to the eighty-first embodiment in a pharmaceutically acceptable solvent or mixture of solvents.

In a ninety-eighth embodiment the invention provides a process for preparing a pharmaceutical composition of Compound 3 comprising dissolving a compound according to the eighty-second embodiment in a pharmaceutically acceptable solvent or mixture of solvents.

In a ninety-ninth embodiment the invention provides a process for preparing a pharmaceutical composition of Compound 3 comprising dissolving a compound according to the eighty-third embodiment in a pharmaceutically acceptable solvent or mixture of solvents.

In a one hundredth embodiment the invention provides a process for preparing a pharmaceutical composition of Compound 3 comprising dissolving a compound according to the eighty-fourth embodiment in a pharmaceutically acceptable solvent or mixture of solvents.

In a one hundred-first embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the seventy-seventh embodiment or (b) a pharmaceutical composition comprising a compound according to the seventy-eighth embodiment and one or more pharmaceutically acceptable excipients.

In a one hundred-second embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the seventy-eighth embodiment or (b) a pharmaceutical composition comprising a compound according to the seventy-eighth embodiment and one or more pharmaceutically acceptable excipients.

In a one hundred-third embodiment the invention provides a method of treating a disease characterized by sphingosine-1-phosphate dysfunction and/or overexpression of sphingosine-1-phosphate, comprising administering to a subject having the disease a therapeutically effective amount of (a) a compound of according to the seventy-ninth embodiment or (b) a pharmaceutical composition comprising a compound according to the seventy-ninth embodiment and one or more pharmaceutically acceptable excipients.

In a one hundred-fourth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the eightieth embodiment or (b) a pharmaceutical composition comprising a compound according to the eightieth embodiment and one or more pharmaceutically acceptable excipients.

In a one hundred-fifth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the eighty-first embodiment or (b) a pharmaceutical composition comprising a compound according to the eighty-first embodiment and one or more pharmaceutically acceptable excipients.

In a one hundred-sixth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the eighty-second embodiment or (b) a pharmaceutical composition comprising a compound according to the eighty-second embodiment and one or more pharmaceutically acceptable excipients.

In a one hundred-seventh embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the eighty-third embodiment or (b) a pharmaceutical composition comprising a compound to the eighty-third embodiment and one or more pharmaceutically acceptable excipients.

In a one hundred-eighth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the eighty-fourth embodiment or (b) a pharmaceutical composition comprising a compound to the eighty-fourth embodiment and one or more pharmaceutically acceptable excipients.

In a one hundred-ninth embodiment the invention provides a method of treating a disease characterized by sphingosine-1-phosphate dysfunction and/or overexpression of sphingosine-1-phosphate, comprising administering to a subject having the disease a therapeutically effective amount of (a) a compound according to the eighty-fifth embodiment or (b) a pharmaceutical composition comprising a compound to the eighty-fifth embodiment and one or more pharmaceutically acceptable excipients.

In a one hundred-tenth embodiment the invention provides a method according to any of the foregoing embodiments wherein the disease is organ transplant rejection, a neoplastic disease or an autoimmune disease.

In a one hundred-eleventh embodiment the invention provides a method according to the one hundred seventh embodiment wherein the organ transplant rejection is kidney transplant rejection, liver transplant rejection, pancreas transplant rejection, small bowel transplant rejection, bone graft rejection or bone marrow transplant (BMT) rejection.

In a one hundred-twelfth embodiment the invention provides a method according to any of the foregoing embodiments wherein the neoplastic disease is selected from the group consisting of cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma, sarcomas, solid tumors, Kaposi's sarcoma, malignant Lymphoma, malignant histiocytosis, malignant melanoma, chronic salicylate intoxication, colorectal carcinoma, multiple myeloma, and combinations thereof.

In a one hundred-thirteenth embodiment the invention provides a method according to any of the foregoing embodiments wherein the autoimmune disease is selected from the group comprising CNS system disorders, multiple sclerosis, arthritis, rheumatoid arthritis, osteoarthritis, ankylosing spondylitis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, acute or chronic immune disease associated with organ transplantation, sarcoidosis, uveitis, systemic lupus, multiple sclerosis, ankylosing spondylitis associated lung disease, acute immune disease associated with organ transplantation, chronic immune disease associated with organ transplantation, rheumatoid spondylitis, juvenile rheumatoid arthritis, or systemic onset juvenile rheumatoid arthritis.

In a one hundred-fourteenth embodiment the invention provides a method according to the one hundred-tenth embodiment wherein the autoimmune disease is MS, arthritis, arheumatoid arthritis, osteoarthritis, ankylosing spondylitis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, psoriasis, multiple sclerosis, rheumatoid spondylitis, juvenile rheumatoid arthritis, or systemic onset juvenile rheumatoid arthritis.

The compounds, compositions comprising the compounds, methods for making the compounds and methods for treating and preventing conditions and disorders by administering the compounds are further described herein.

### DETAILED DESCRIPTION OF THE INVENTION

In various embodiments, salts and crystalline forms of Compound 1, 2 or 3 are provided. Crystalline forms include solvates, hydrates, anhydrates, and salts of Compound 1, 2 or 3.

In contrast to an amorphous form of Compound 1, 2 or 3 free acid and an amorphous form of a Compound 1, 2 or 3, a salt or a crystalline form is characterized by the presence of observable peaks in a powder x-ray diffraction (PXRD) pattern measured on the crystalline form. For crystalline forms prepared to yield suitably sized single-crystals, the crystalline form can be further characterized through an experimental determination of the unit cell parameters, the identification of the crystallographic space group to which a single crystal belongs, or both of these. Once the unit cell parameters are known, the location of the diffraction peaks, and in particular the 2θ values of the peaks in a PXRD pattern can be calculated, to further characterize the crystalline form. The PXRD pattern can also be measured experimentally for such crystalline forms. The PXRD patterns measured or calculated for the salts and crystalline forms reported herein represent a fingerprint that can be compared to other experimentally determined patterns to find a match. Identity of the respective crystalline forms is established by overlap or match of an experimentally determined PXRD pattern with the PXRD pattern of the crystalline forms reported herein. In various embodiments, the salts and crystalline forms are characterized by exhibiting at least one of the PXRD peaks reported here. Thus, in various embodiments, a salt or crystalline form is characterized by a match of one or more peaks, two or more peaks, three or more peaks, four or more peaks, or five or more peaks, and so on, from the respective PXRD patterns.

An embodiment of the synthesis of compounds of the invention (free acids) and representative intermediate compounds are presented below. The exemplified compounds are named using ACD/ChemSketch Version 5.06 (5 Jun. 2001, Advanced Chemistry Development Inc., Toronto, Ontario), ACD/ChemSketch Version 12.01 (13 May 2009), Advanced Chemistry Development Inc., Toronto, Ontario), or ChemDraw® Ver. 9.0.5 (CambridgeSoft, Cambridge, Mass.). Intermediates are named using ChemDraw® Ver. 9.0.5 (CambridgeSoft, Cambridge, Mass.).

A sphingosine-1-phosphate-1 (S1P₁) receptor agonist, referred to herein as Compound 1, has the systematic name 3-{3-chloro-4-[5-(5-chloro-6-isopropoxy-pyridin-3-yl)-[1,2,4]oxadiazol-3-yl]-phenoxy}-cyclobutanecarboxylic acid, and can be depicted by the formula:

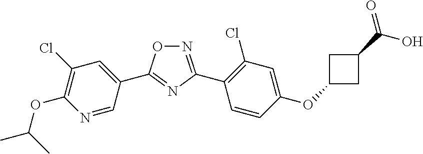

Compounds of the invention include salts and crystalline forms of 3-{3-chloro-4-[5-(5-chloro-6-isopropoxy-pyridin-3-yl)-[1,2,4]oxadiazol-3-yl]-phenoxy}-cyclobutanecarboxylic acid active agent.

It has also been discovered that 3-{3-chloro-4-[5-(5-chloro-6-isopropoxy-pyridin-3-yl)-[1,2,4]oxadiazol-3-yl]-phenoxy}-cyclobutanecarboxylic acid salts can be prepared as new crystalline forms.

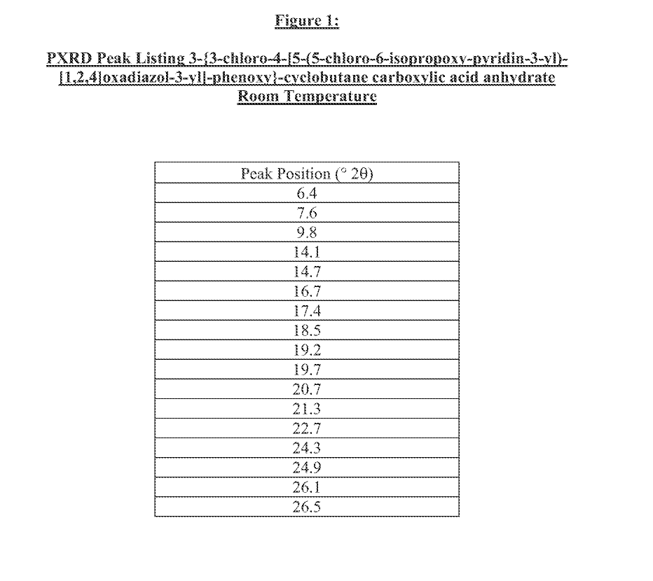

3-{3-chloro-4-[5-(5-chloro-6-isopropoxy-pyridin-3-yl)-[1,2,4]oxadiazol-3-yl]-phenoxy}-cyclobutane carboxylic acid anhydrate can be identified by its powder X-ray diffraction pattern in accordance with the Brief Description of the Drawings (FIG. 1). Powder X-ray diffraction peak listing is also shown in FIG. 1.

Crystallographic information of 3-{3-chloro-4-[5-(5-chloro-6-isopropoxy-pyridin-3-yl)-[1,2,4]oxadiazol-3-yl]-phenoxy}-cyclobutane carboxylic acid anhydrate is shown in FIG. 1A.

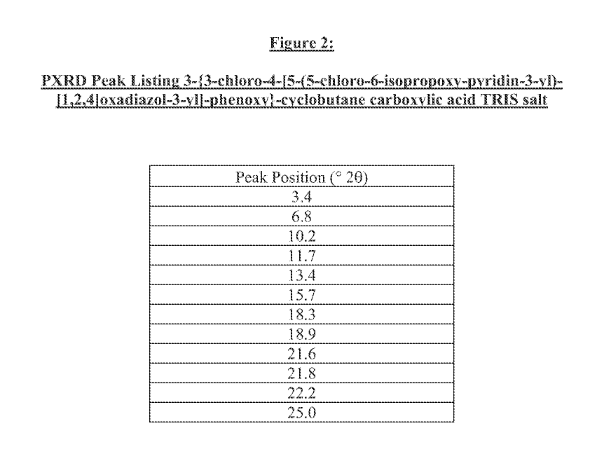

3-{3-chloro-4-[5-(5-chloro-6-isopropoxy-pyridin-3-yl)-[1,2,4]oxadiazol-3-yl]-phenoxy}-cyclobutane carboxylic acid TRIS salt can be identified by its powder X-ray diffraction pattern in accordance with the Brief Description of the Drawings (FIG. 2). Powder X-ray diffraction peak listing is also shown in FIG. 2.

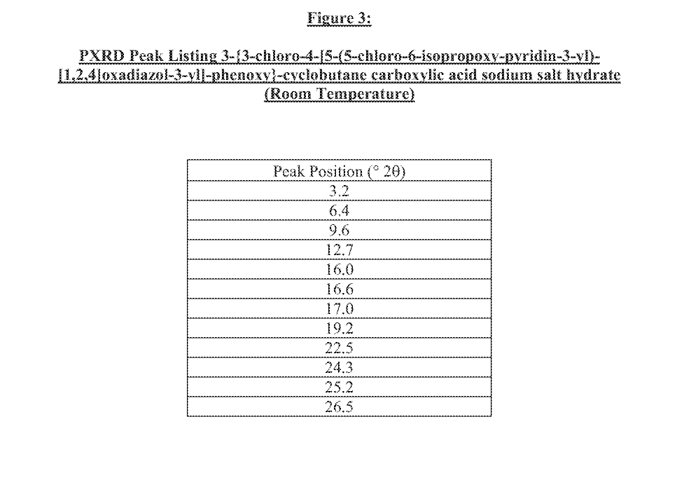

3-{3-chloro-4-[5-(5-chloro-6-isopropoxy-pyridin-3-yl)-[1,2,4]oxadiazol-3-yl]-phenoxy}-cyclobutane carboxylic acid sodium salt hydrate can be identified by its powder X-ray diffraction pattern in accordance with the Brief Description of the Drawings (FIG. 3). Powder X-ray diffraction peak listing is also shown in FIG. 3.

Crystallographic information of 3-{3-chloro-4-[5-(5-chloro-6-isopropoxy-pyridin-3-yl)-[1,2,4]oxadiazol-3-yl]-phenoxy}-cyclobutane carboxylic acid sodium salt hydrate is shown in FIG. 3A.

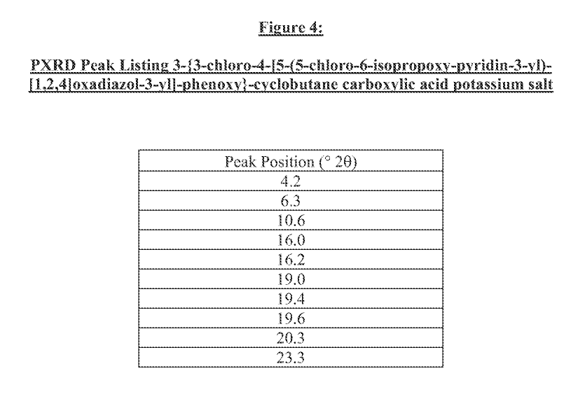

3-{3-chloro-4-[5-(5-chloro-6-isopropoxy-pyridin-3-yl)-[1,2,4]oxadiazol-3-yl]-phenoxy}-cyclobutane carboxylic acid potassium salt can be identified by its powder X-ray diffraction pattern in accordance with the Brief Description of the Drawings (FIG. 4). Powder X-ray diffraction peak listing is also shown in FIG. 4.

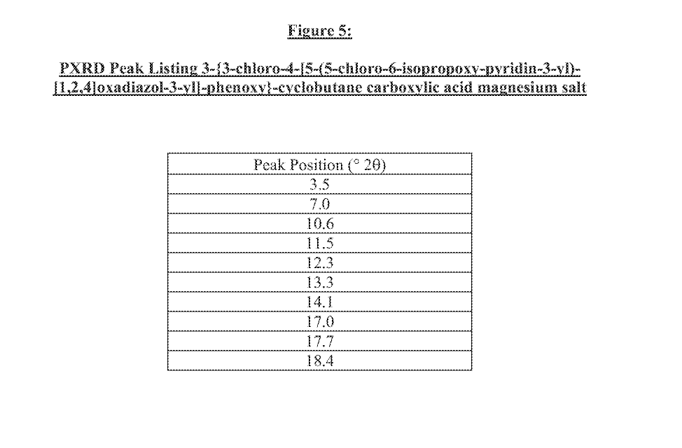

3-{3-chloro-4-[5-(5-chloro-6-isopropoxy-pyridin-3-yl)-[1,2,4]oxadiazol-3-yl]-phenoxy}-cyclobutane carboxylic acid magnesium salt can be identified by its powder X-ray diffraction pattern in accordance with the Brief Description of the Drawings (FIG. 5). Powder X-ray diffraction peak listing is also shown in FIG. 5.

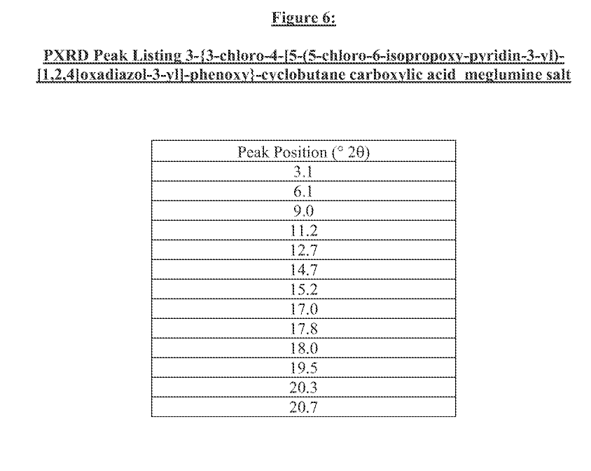

3-{3-chloro-4-[5-(5-chloro-6-isopropoxy-pyridin-3-yl)-[1,2,4]oxadiazol-3-yl]-phenoxy}-cyclobutane carboxylic acid meglumine salt can be identified by its powder X-ray diffraction pattern in accordance with the Brief Description of the Drawings (FIG. 6). Powder X-ray diffraction peak listing is also shown in FIG. 6.

A second sphingosine-1-phosphate-1 (S1P₁) receptor agonist, referred to herein as Compound 2, has the systematic name 3-(2-(4-(3-(3-chloro-4-isopropoxyphenyl)-1,2,4-oxadiazol-5-yl)phenyl)propan-2-ylamino)propanoic acid, and can be depicted by the formula:

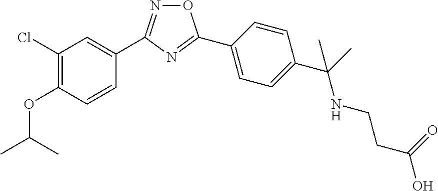

In various embodiments, salts and crystalline forms of Compound 2 are provided. Crystalline forms include solvates, hydrates, anhydrates, and salts of Compound 2.

Compounds of the invention include salts, solvates and crystalline forms of 3-(2-(4-(3-(3-chloro-4-isopropoxyphenyl)-1,2,4-oxadiazol-5-yl)phenyl)propan-2-ylamino)propanoic acid active agent.

It has also been discovered that 3-(2-(4-(3-(3-chloro-4-isopropoxyphenyl)-1,2,4-oxadiazol-5-yl)phenyl)propan-2-ylamino)propanoic acid salts can be prepared as new crystalline forms.

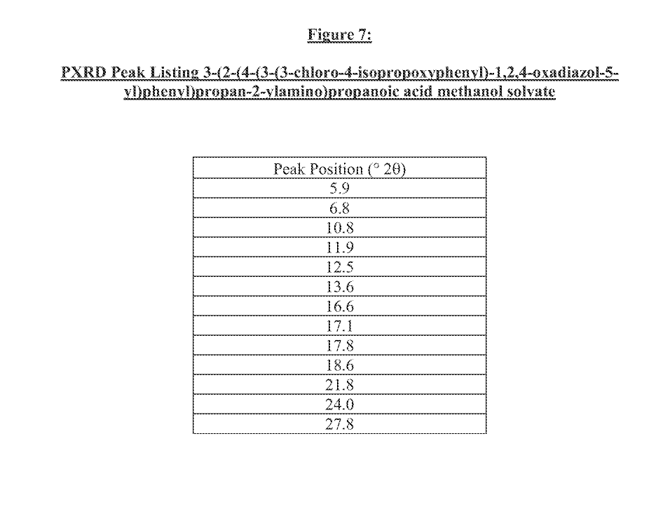

3-(2-(4-(3-(3-chloro-4-isopropoxyphenyl)-1,2,4-oxadiazol-5-yl)phenyl)propan-2-ylamino)propanoic acid methanol solvate can be identified by its powder X-ray diffraction pattern in accordance with the Brief Description of the Drawings (FIG. 7). Powder X-ray diffraction peak listing is also shown in FIG. 7.

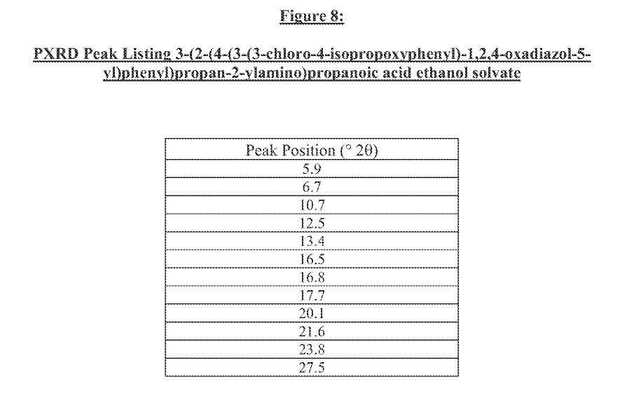

3-(2-(4-(3-(3-chloro-4-isopropoxyphenyl)-1,2,4-oxadiazol-5-yl)phenyl)propan-2-ylamino)propanoic acid ethanol solvate can be identified by its powder X-ray diffraction pattern in accordance with the Brief Description of the Drawings (FIG. 8). Powder X-ray diffraction peak listing is also shown in FIG. 8.

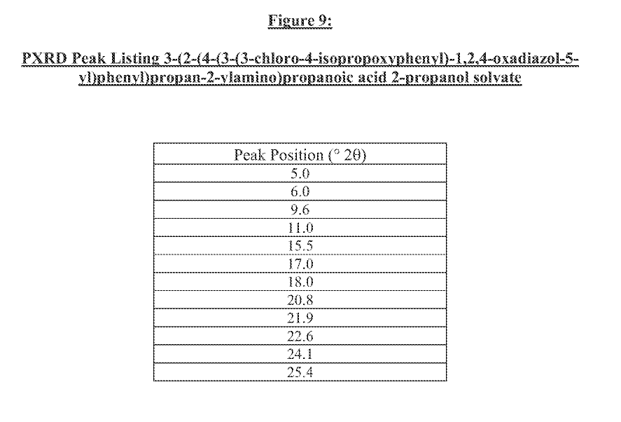

3-(2-(4-(3-(3-chloro-4-isopropoxyphenyl)-1,2,4-oxadiazol-5-yl)phenyl)propan-2-ylamino)propanoic acid 2-propanol solvate can be identified by its powder X-ray diffraction pattern in accordance with the Brief Description of the Drawings (FIG. 9). Powder X-ray diffraction peak listing is also shown in FIG. 9.

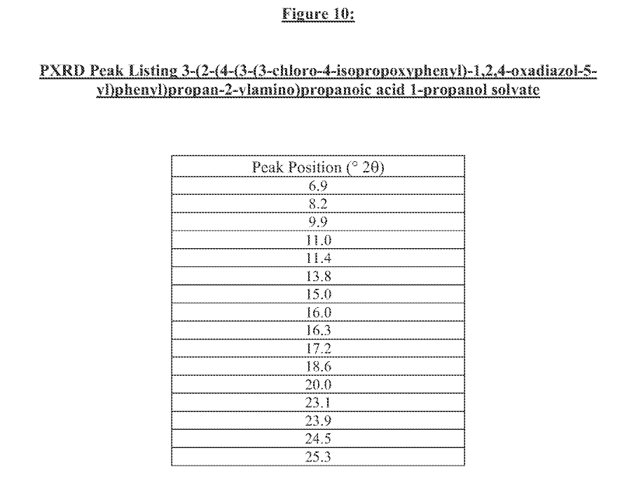

3-(2-(4-(3-(3-chloro-4-isopropoxyphenyl)-1,2,4-oxadiazol-5-yl)phenyl)propan-2-ylamino)propanoic acid 1-propanol solvate can be identified by its powder X-ray diffraction pattern in accordance with the Brief Description of the Drawings (FIG. 10). Powder X-ray diffraction peak listing is also shown in FIG. 10.

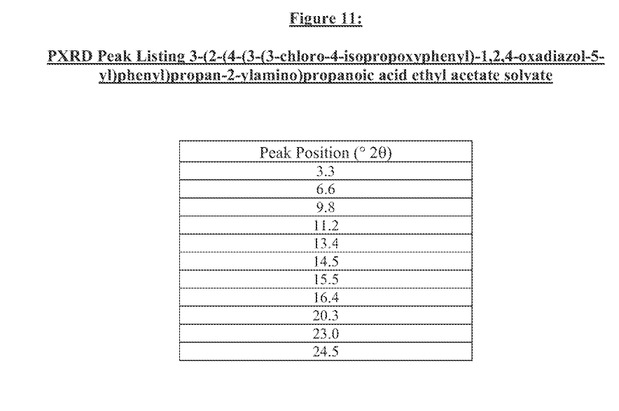

3-(2-(4-(3-(3-chloro-4-isopropoxyphenyl)-1,2,4-oxadiazol-5-yl)phenyl)propan-2-ylamino)propanoic acid ethyl acetate solvate can be identified by its powder X-ray diffraction pattern in accordance with the Brief Description of the Drawings (FIG. 11). Powder X-ray diffraction peak listing is also shown in FIG. 11.

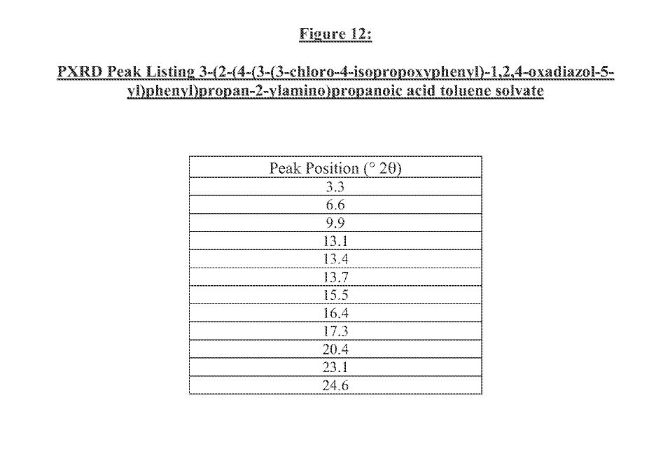

3-(2-(4-(3-(3-chloro-4-isopropoxyphenyl)-1,2,4-oxadiazol-5-yl)phenyl)propan-2-ylamino)propanoic acid tolulene solvate can be identified by its powder X-ray diffraction pattern in accordance with the Brief Description of the Drawings (FIG. 12). Powder X-ray diffraction peak listing is also shown in FIG. 12.

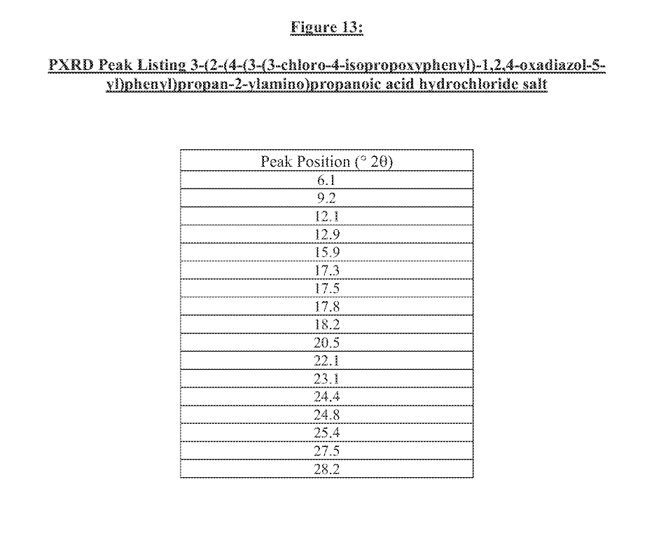

3-(2-(4-(3-(3-chloro-4-isopropoxyphenyl)-1,2,4-oxadiazol-5-yl)phenyl)propan-2-ylamino)propanoic acid hydrochloride salt can be identified by its powder X-ray diffraction pattern in accordance with the Brief Description of the Drawings (FIG. 13). Powder X-ray diffraction peak listing is also shown in FIG. 13.

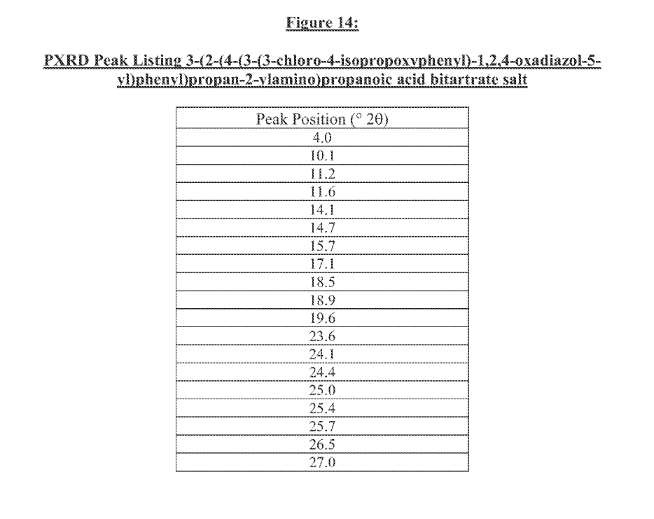

3-(2-(4-(3-(3-chloro-4-isopropoxyphenyl)-1,2,4-oxadiazol-5-yl)phenyl)propan-2-ylamino)propanoic acid bitartrate salt can be identified by its powder X-ray diffraction pattern in accordance with the Brief Description of the Drawings (FIG. 14). Powder X-ray diffraction peak listing is also shown in FIG. 14.

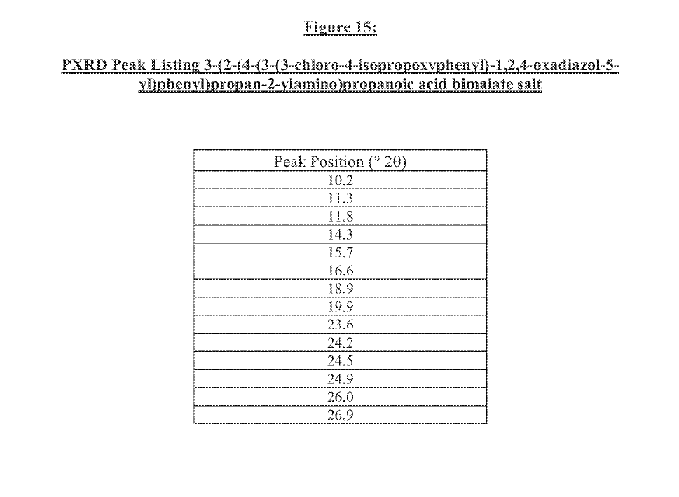

3-(2-(4-(3-(3-chloro-4-isopropoxyphenyl)-1,2,4-oxadiazol-5-yl)phenyl)propan-2-ylamino)propanoic acid bimalate salt can be identified by its powder X-ray diffraction pattern in accordance with the Brief Description of the Drawings (FIG. 15). Powder X-ray diffraction peak listing is also shown in FIG. 15.

A third sphingosine-1-phosphate-1 (S1P₁) receptor agonist, referred to herein as Compound 3, has the systematic name (1R,3S)-3-(4-(5-(5-chloro-6-isopropoxypyridin-3-yl)-1,2,4-oxadiazol-3-yl)phenylamino)cyclopentanecarboxylic acid, and can be depicted by the formula:

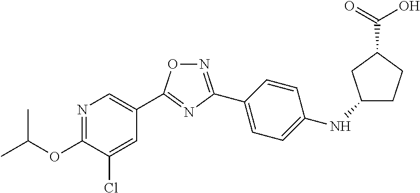

In various embodiments, salts and crystalline forms of Compound 3 are provided. Crystalline forms include solvates, hydrates, anhydrates, and salts of Compound 3.

Compounds of the invention include salts, solvates and crystalline forms of (1R,3S)-3-(4-(5-(5-chloro-6-isopropoxypyridin-3-yl)-1,2,4-oxadiazol-3-yl)phenylamino)cyclopentanecarboxylic acid active agent.

It has also been discovered that (1R,3S)-3-(4-(5-(5-chloro-6-isopropoxypyridin-3-yl)-1,2,4-oxadiazol-3-yl)phenylamino)cyclopentanecarboxylic acid salts can be prepared as new crystalline forms.

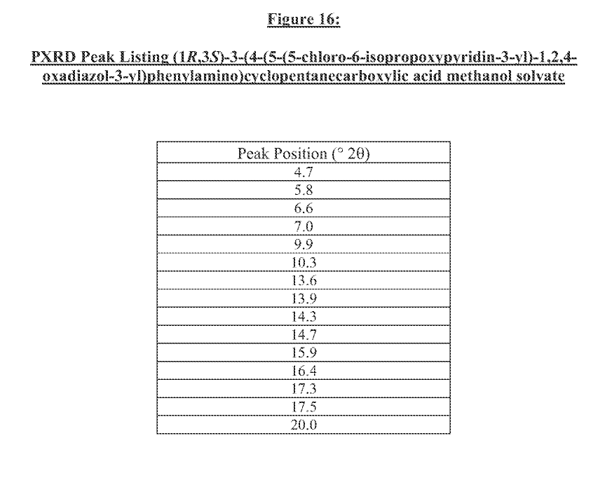

(1R,3S)-3-(4-(5-(5-chloro-6-isopropoxypyridin-3-yl)-1,2,4-oxadiazol-3-yl)phenylamino)cyclopentanecarboxylic acid methanol solvate can be identified by its powder X-ray diffraction pattern in accordance with the Brief Description of the Drawings (FIG. 16). Powder X-ray diffraction peak listing is also shown in FIG. 16.

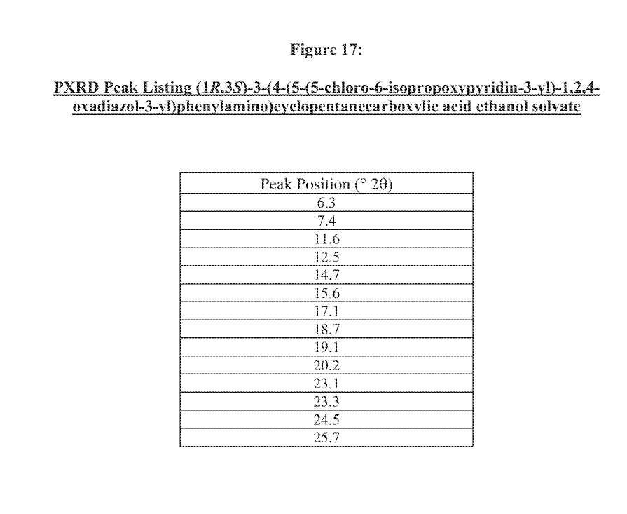

(1R,3S)-3-(4-(5-(5-chloro-6-isopropoxypyridin-3-yl)-1,2,4-oxadiazol-3-yl)phenylamino)cyclopentanecarboxylic acid ethanol solvate can be identified by its powder X-ray diffraction pattern in accordance with the Brief Description of the Drawings (FIG. 17). Powder X-ray diffraction peak listing is also shown in FIG. 17.

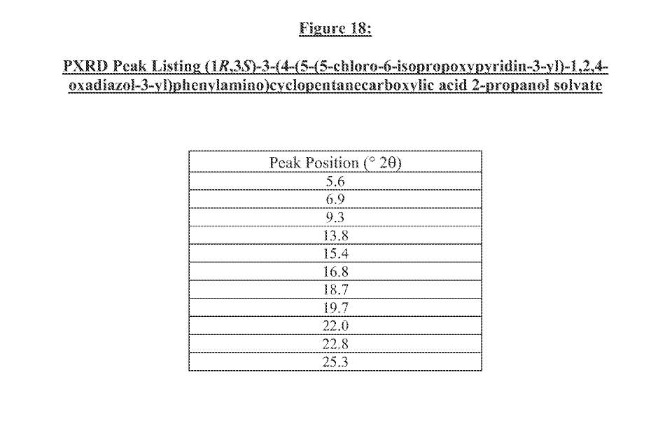

(1R,3S)-3-(4-(5-(5-chloro-6-isopropoxypyridin-3-yl)-1,2,4-oxadiazol-3-yl)phenylamino)cyclopentanecarboxylic acid 2-propanol solvate can be identified by its powder X-ray diffraction pattern in accordance with the Brief Description of the Drawings (FIG. 18). Powder X-ray diffraction peak listing is also shown in FIG. 18.

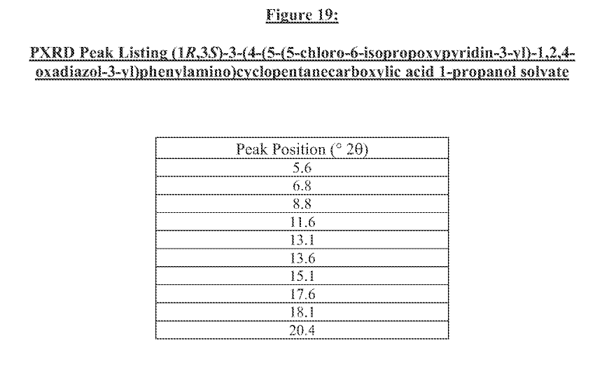

(1R,3S)-3-(4-(5-(5-chloro-6-isopropoxypyridin-3-yl)-1,2,4-oxadiazol-3-yl)phenylamino)cyclopentanecarboxylic acid 1-propanol solvate can be identified by its powder X-ray diffraction pattern in accordance with the Brief Description of the Drawings (FIG. 19). Powder X-ray diffraction peak listing is also shown in FIG. 19.

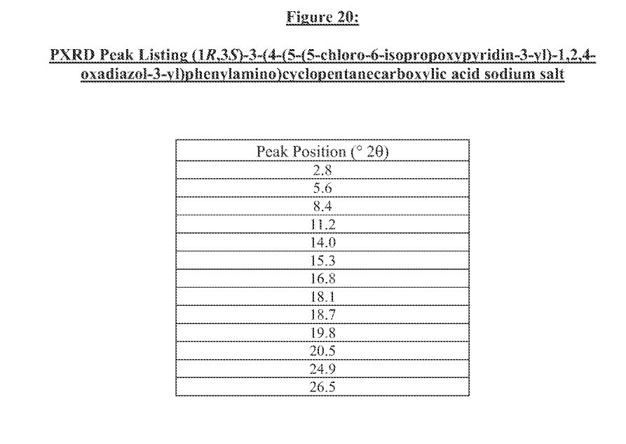

(1R,3S)-3-(4-(5-(5-chloro-6-isopropoxypyridin-3-yl)-1,2,4-oxadiazol-3-yl)phenylamino)cyclopentanecarboxylic acid sodium salt can be identified by its powder X-ray diffraction pattern in accordance with the Brief Description of the Drawings (FIG. 20). Powder X-ray diffraction peak listing is also shown in FIG. 20.

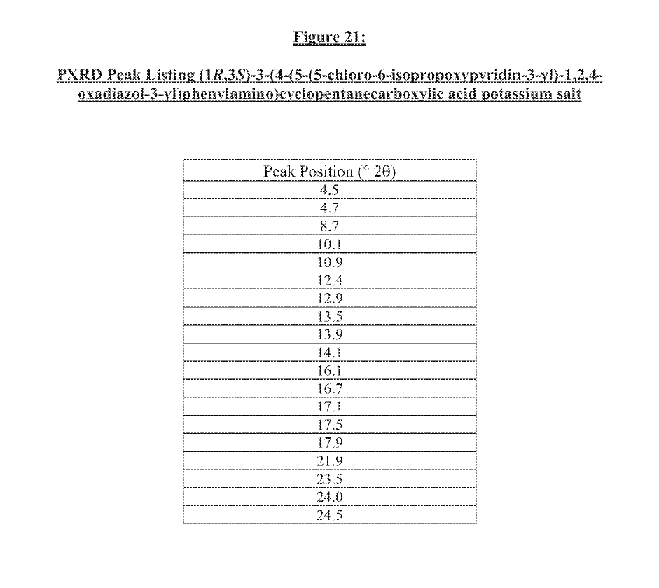

(1R,3S)-3-(4-(5-(5-chloro-6-isopropoxypyridin-3-yl)-1,2,4-oxadiazol-3-yl)phenylamino)cyclopentanecarboxylic acid potassium salt can be identified by its powder X-ray diffraction pattern in accordance with the Brief Description of the Drawings (FIG. 21). Powder X-ray diffraction peak listing is also shown in FIG. 21.

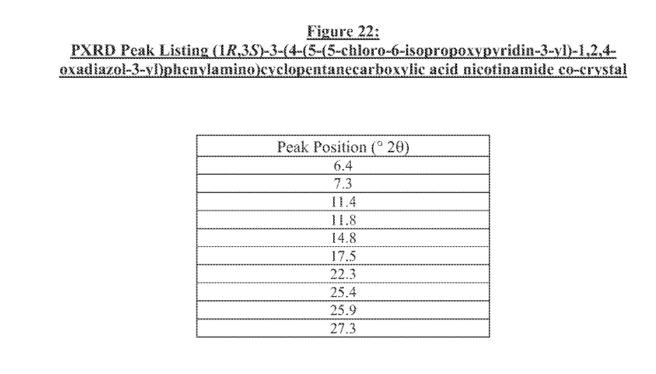

(1R,3S)-3-(4-(5-(5-chloro-6-isopropoxypyridin-3-yl)-1,2,4-oxadiazol-3-yl)phenylamino)cyclopentanecarboxylic acid nicotinamide co-crystal can be identified by its powder X-ray diffraction pattern in accordance with the Brief Description of the Drawings (FIG. 22). Powder X-ray diffraction peak listing is also shown in FIG. 22.

### Methods of Use

The present invention provides salts, solvates, hydrates, anhydrates and crystalline forms of 3-{3-chloro-4-[5-(5-chloro-6-isopropoxy-pyridin-3-yl)-[1,2,4]oxadiazol-3-yl]-phenoxy}-cyclobutane carboxylic acid, 3-(2-(4-(3-(3-chloro-4-isopropoxyphenyl)-1,2,4-oxadiazol-5-yl)phenyl)propan-2-ylamino)propanoic acid and (1R,3S)-3-(4-(5-(5-chloro-6-isopropoxypyridin-3-yl)-1,2,4-oxadiazol-3-yl)phenylamino)cyclopentanecarboxylic acid which are effective as antagonists or agonists of the G protein-coupled S1P receptor family. These compounds reduce the number of circulating and infiltrating T- and B-lymphocytes, affording a beneficial immunosuppressive effect.

The present invention also provides compounds that exhibit activity within the S1P receptor family.

In a related aspect the invention provides a method for modulating receptors of the S1P family in a human subject suffering from a disorder in which modulation of S1P activity is beneficial, comprising administering to the human subject a compound of Formula I such that modulation of S1P activity in the human subject is triggered and treatment is achieved.

In another related aspect the invention provides a method of modulating sphingosine-1-phosphate receptor 1 (S1P₁) activity comprising contacting a cell with one or more compounds of the invention.

A compound of the invention or pharmaceutical compositions containing a therapeutically effective amount thereof is useful in the treatment of a disorder selected from the group comprising CNS system disorders, multiple sclerosis, arthritis, rheumatoid arthritis, osteoarthritis, juvenile chronic arthritis, Lyme arthritis, psoriatic arthritis, reactive arthritis, osteoarthritis, septic arthritis, spondyloarthropathy, systemic lupus erythematosus, Crohn's disease, ulcerative colitis, inflammatory bowel disease, ankylosing spondylitis, insulin dependent diabetes mellitus, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, Parkinson's disease, Alzheimer's disease, stroke, ulcerative colitic arthropathy, rheumatoid arthritis associated interstitial lung disease, systemic lupus erythematosus associated lung disease, dermatomyositis/polymyositis associated lung disease, ankylosing spondylitis associated lung disease, gouty arthritis, acute immune disease associated with organ transplantation, chronic immune disease associated with organ transplantation, discoid lupus erythematosus, male infertility idiopathic or NOS, multiple sclerosis (all subtypes), rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, cystic fibrosis, juvenile rheumatoid arthritis, juvenile spinal muscular atrophy, systemic inflammatory response syndrome, systemic onset juvenile rheumatoid arthritis, In addition, such compounds may be useful in the treatment of proliferative disorders such as restenosis, or a central nervous system disorder.

In the compositions of the present invention the active compound may, if desired, be associated with other compatible pharmacologically active ingredients. For example, the compounds of this invention can be administered in combination with another therapeutic agent that is known to treat a disease or condition described herein.

One or more compounds of the invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

### Pharmaceutical Compositions and Modes of Administration

Suitable routes of administration may, for example, include oral, eyedrop, rectal, transmucosal, topical, or intestinal administration, parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoro-methane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 400, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

### Dosage

For any compound used in a method of the present invention, the therapeutically effective dose can be estimated initially from cellular assays. For example, a dose can be formulated in cellular and animal models to achieve a circulating concentration range that includes the EC₅₀ as determined in cellular assays (i.e., the concentration of the test compound which achieves a half-maximal inhibition of a given receptor activity). In some cases it is appropriate to determine the EC₅₀ in the presence of 3 to 5% serum albumin since such a determination approximates the binding effects of plasma protein on the compound. Such information can be used to more accurately determine useful doses in humans. Further, advantageous compounds for systemic administration effectively modulate receptors of the S1P family in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1, p. 1). In the treatment of crises, the administration of an acute bolus or an infusion approaching the MTD may be advantageous to obtain a rapid response.

Dosage amount and interval may be adjusted individually to provide plasma levels of the active moiety which are sufficient to modulate receptors of the S1P family, or minimal effective concentration (MEC). The MEC will vary for each compound but can be estimated from in vitro data; e.g. the concentration necessary to achieve 50-90% inhibition of binding of the natural ligand using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and more preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

### Exemplary Formulations

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

### a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

### b) Tablets

Tablets can be prepared, for example, from the following ingredients:

### Parts by Weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

The present invention also comprises the use of a compound of Formula I as a medicament.

The teachings of all references, including journal articles, patents and published patent applications, are incorporated herein by reference in their entirety.

### Example 1

### 3-{3-chloro-4-[5-(5-chloro-6-isopropoxy-pyridin-3-yl)-[1,2,4]oxadiazol-3-yl]-phenoxy}-cyclobutane carboxylic acid anhydrate

Water saturated ethyl acetate was saturated with solid 3-{3-chloro-4-[5-(5-chloro-6-isopropoxy-pyridin-3-yl)-[1,2,4]oxadiazol-3-yl]-phenoxy}-cyclobutane carboxylic acid. The suspension was filtered and the solution was allowed to evaporate. Single crystals of 3-{3-chloro-4-[5-(5-chloro-6-isopropoxy-pyridin-3-yl)-[1,2,4]oxadiazol-3-yl]-phenoxy}-cyclobutane carboxylic acid were observed upon evaporation.

Powder X-ray diffraction pattern, peak listing, and crystallographic information can be seen in FIG. 1.

### Example 2

### 3-{3-chloro-4-[5-(5-chloro-6-isopropoxy-pyridin-3-yl)-[1,2,4]oxadiazol-3-yl]-phenoxy}-cyclobutane carboxylic acid TRIS salt

### Example 2A

3-{3-chloro-4-[5-(5-chloro-6-isopropoxy-pyridin-3-yl)-[1,2,4]oxadiazol-3-yl]-phenoxy}-cyclobutane carboxylic acid solids (77 mg) were suspended in acetonitrile (1.75 mL) at about 40° C. TRIS solids (23 mg) were dissolved in water (0.25 mL). TRIS solution was then added to the above 3-{3-chloro-4-[5-(5-chloro-6-isopropoxy-pyridin-3-yl)-[1,2,4]oxadiazol-3-yl]-phenoxy}-cyclobutane carboxylic acid suspension. The resulting suspension was sonicated for about 10 minutes and magnetically stirred overnight.

### Example 2B

3-{3-chloro-4-[5-(5-chloro-6-isopropoxy-pyridin-3-yl)-[1,2,4]oxadiazol-3-yl]-phenoxy}-cyclobutane carboxylic acid solids (95 mg) were suspended in acetonitrile (1.70 mL) at about 40° C. TRIS solids (28 mg) were dissolved in water (0.30 mL). TRIS solution was then added to the above 3-{3-chloro-4-[5-(5-chloro-6-isopropoxy-pyridin-3-yl)-[1,2,4]oxadiazol-3-yl]-phenoxy}-cyclobutane carboxylic acid suspension. The resulting suspension was magnetically stirred for about one hour.

Powder X-ray diffraction pattern and peak listing is shown in FIG. 2.

### Example 3

### 3-{3-chloro-4-[5-(5-chloro-6-isopropoxy-pyridin-3-yl)-[1,2,4]oxadiazol-3-yl]-phenoxy}-cyclobutane carboxylic acid sodium salt hydrate

### Example 3A

3-{3-chloro-4-[5-(5-chloro-6-isopropoxy-pyridin-3-yl)-[1,2,4]oxadiazol-3-yl]-phenoxy}-cyclobutane carboxylic acid solids (48 mg) were suspended in acetonitrile (10.0 mL) at about 60° C. Aqueous sodium hydroxide solution (62 μL, 50% w/w,) and water (50 μL) were added to the 3-{3-chloro-4-[5-(5-chloro-6-isopropoxy-pyridin-3-yl)-[1,2,4]oxadiazol-3-yl]-phenoxy}-cyclobutane carboxylic acid suspension.

### Example 3B

3-{3-chloro-4-[5-(5-chloro-6-isopropoxy-pyridin-3-yl)-[1,2,4]oxadiazol-3-yl]-phenoxy}-cyclobutane carboxylic acid solids (500 mg) was suspended in acetonitrile/water mixture (10 mL, 95/5 v/v) at 60° C. Aqueous sodium hydroxide solution (62 μL, 50% w/w) was added to the 3-{3-chloro-4-[5-(5-chloro-6-isopropoxypyridin-3-yl)-[1,2,4]oxadiazol-3-yl]-phenoxy}-cyclobutane carboxylic acid suspension. The suspension was then sonicated for about 20 minutes.

Powder X-ray diffraction pattern, peak listing, and crystallographic information can be seen in FIG. 3.

### Example 4

### 3-{3-chloro-4-[5-(5-chloro-6-isopropoxy-pyridin-3-yl)-[1,2,4]oxadiazol-3-yl]-phenoxy}-cyclobutane carboxylic acid potassium salt

3-{3-chloro-4-[5-(5-chloro-6-isopropoxy-pyridin-3-yl)-[1,2,4]oxadiazol-3-yl]-phenoxy}-cyclobutane carboxylic acid solids (20 mg) were suspended in acetonitrile (0.15 mL). Methanol potassium hydroxide solution (0.05 mL, 1M) was added to the above 3-{3-chloro-4-[5-(5-chloro-6-isopropoxy-pyridin-3-yl)-[1,2,4]oxadiazol-3-yl]-phenoxy}-cyclobutane carboxylic acid suspension. Solids initially began to dissolve and then crystallization was observed.

Powder X-ray diffraction pattern and peak listing is shown in FIG. 4.

### Example 5

### 3-{3-chloro-4-[5-(5-chloro-6-isopropoxy-pyridin-3-yl)-[1,2,4]oxadiazol-3-yl]-phenoxy}-cyclobutane carboxylic acid magnesium salt

3-{3-chloro-4-[5-(5-chloro-6-isopropoxy-pyridin-3-yl)-[1,2,4]oxadiazol-3-yl]-phenoxy}-cyclobutane carboxylic acid solids (25 mg) and magnesium hydroxide solids (4 mg) were suspended in acetonitrile (0.5 mL) overnight. Water (0.1 mL) was added to the above suspension. Caking was observed. The resulting solids were filtered and analyzed.

Powder X-ray diffraction pattern and peak listing is shown in FIG. 5.

### Example 6

### 3-{3-chloro-4-[5-(5-chloro-6-isopropoxy-pyridin-3-yl)-[1,2,4]oxadiazol-3-yl]-phenoxy}-cyclobutane carboxylic acid meglumine salt

3-{3-chloro-4-[5-(5-chloro-6-isopropoxy-pyridin-3-yl)-[1,2,4]oxadiazol-3-yl]-phenoxy}-cyclobutane carboxylic acid solids (25 mg) and meglumine solids (12 mg) were suspended in acetonitrile (0.5 mL). Caking was observed. The resulting solids were filtered and analyzed.

Powder X-ray diffraction pattern and peak listing is shown in FIG. 6.

### Example 7

### 3-(2-(4-(3-(3-chloro-4-isopropoxyphenyl)-1,2,4-oxadiazol-5-yl)phenyl)propan-2-ylamino)propanoic acid methanol solvate

3-(2-(4-(3-(3-Chloro-4-isopropoxyphenyl)-1,2,4-oxadiazol-5-yl)phenyl)propan-2-ylamino)propanoic acid solids (27 mg) were suspended in methanol (0.35 mL) at about 25° C. The suspension was magnetically stirred for about 3 days. The resulting solids were filtered and analyzed.

Powder X-ray diffraction pattern and peak listing is shown in FIG. 7.

### Example 8

### 3-(2-(4-(3-(3-chloro-4-isopropoxyphenyl)-1,2,4-oxadiazol-5-yl)phenyl)propan-2-ylamino)propanoic acid ethanol solvate

3-(2-(4-(3-(3-Chloro-4-isopropoxyphenyl)-1,2,4-oxadiazol-5-yl)phenyl)propan-2-ylamino)propanoic acid solids (28 mg) were suspended in ethanol (0.5 mL) at about 25° C. The suspension was magnetically stirred for about 3 days. The resulting solids were filtered and analyzed.

Powder X-ray diffraction pattern and peak listing is shown in FIG. 8.

### Example 9

### 3-(2-(4-(3-(3-chloro-4-isopropoxyphenyl)-1,2,4-oxadiazol-5-yl)phenyl)propan-2-ylamino)propanoic acid 2-propanol solvate

3-(2-(4-(3-(3-Chloro-4-isopropoxyphenyl)-1,2,4-oxadiazol-5-yl)phenyl)propan-2-ylamino)propanoic acid solids (30 mg) were suspended in 2-propanol (1.0 mL) at about 25° C. The suspension was magnetically stirred for about 3 days. The resulting solids were filtered and analyzed.

Powder X-ray diffraction pattern and peak listing is shown in FIG. 9.

### Example 10

### 3-(2-(4-(3-(3-chloro-4-isopropoxyphenyl)-1,2,4-oxadiazol-5-yl)phenyl)propan-2-ylamino)propanoic acid 1-propanol solvate

3-(2-(4-(3-(3-Chloro-4-isopropoxyphenyl)-1,2,4-oxadiazol-5-yl)phenyl)propan-2-ylamino)propanoic acid solids (28 mg) were suspended in 1-propanol (1.0 mL) at about 25° C. The suspension was magnetically stirred for about 3 days. The resulting solids were filtered and analyzed.

Powder X-ray diffraction pattern and peak listing is shown in FIG. 10.

### Example 11

### 3-(2-(4-(3-(3-chloro-4-isopropoxyphenyl)-1,2,4-oxadiazol-5-yl)phenyl)propan-2-ylamino)propanoic acid ethyl acetate solvate

3-(2-(4-(3-(3-Chloro-4-isopropoxyphenyl)-1,2,4-oxadiazol-5-yl)phenyl)propan-2-ylamino)propanoic acid solids (30 mg) were suspended in ethyl acetate (1.0 mL) at about 25° C. The suspension was magnetically stirred for about 3 days. The resulting solids were filtered and analyzed.

Powder X-ray diffraction pattern and peak listing is shown in FIG. 11.

### Example 12

### 3-(2-(4-(3-(3-chloro-4-isopropoxyphenyl)-1,2,4-oxadiazol-5-yl)phenyl)propan-2-ylamino)propanoic acid toluene solvate

3-(2-(4-(3-(3-Chloro-4-isopropoxyphenyl)-1,2,4-oxadiazol-5-yl)phenyl)propan-2-ylamino)propanoic solids (30 mg) were suspended in toluene (0.1 mL) at about 25° C. The suspension was magnetically stirred for about 3 days. The resulting solids were filtered and analyzed.

Powder X-ray diffraction pattern and peak listing is shown in FIG. 12.

### Example 13

### 3-(2-(4-(3-(3-chloro-4-isopropoxyphenyl)-1,2,4-oxadiazol-5-yl)phenyl)propan-2-ylamino)propanoic hydrochloride

3-(2-(4-(3-(3-Chloro-4-isopropoxyphenyl)-1,2,4-oxadiazol-5-yl)phenyl)propan-2-ylamino)propanoic acid solids (25 mg) were suspended in methanol (0.15 mL). Hydrochloric acid (60 μL, 1M, aqueous) was added and all solids were dissolved. Crystallization was observed in less than one hour. The resulting solids were filtered and analyzed.

Powder X-ray diffraction pattern and peak listing is shown in FIG. 13.

### Example 14

### 3-(2-(4-(3-(3-chloro-4-isopropoxyphenyl)-1,2,4-oxadiazol-5-yl)phenyl)propan-2-ylamino)propanoic bitartrate

### Example 14A

3-(2-(4-(3-(3-Chloro-4-isopropoxyphenyl)-1,2,4-oxadiazol-5-yl)phenyl)propan-2-ylamino)propanoic acid solids (25 mg) were suspended in methanol (0.15 mL). L-tartaric acid (60 μL, 1M, aqueous) was added and all solids were dissolved. Crystallization was observed in less than one hour. The resulting solids were filtered and analyzed.

### Example 14B

3-(2-(4-(3-(3-Chloro-4-isopropoxyphenyl)-1,2,4-oxadiazol-5-yl)phenyl)propan-2-ylamino)propanoic acid solids (289 mg) were suspended in 2-propanol/water (90/10, v/v, 2.0 mL). L-tartaric acid (0.7 mL, 1M, aqueous) was added and crystallization was observed. Additional 2-propanol (2.0 mL) was added. The resulting solids were filtered and analyzed.

Powder X-ray diffraction pattern and peak listing is shown in Figure.

### Example 15

### 3-(2-(4-(3-(3-chloro-4-isopropoxyphenyl)-1,2,4-oxadiazol-5-yl)phenyl)propan-2-ylamino)propanoic bimalate

### Example 15A

3-(2-(4-(3-(3-Chloro-4-isopropoxyphenyl)-1,2,4-oxadiazol-5-yl)phenyl)propan-2-ylamino)propanoic acid solids (26 mg) were suspended in methanol (0.15 mL). L-malic acid (70 μL, 1M, aqueous) and water (0.3 mL) was added and the suspension was heated to about 50° C. for about 30 minutes then removed from heat. The resulting solids were filtered and analyzed five days later.

### Example 15B

3-(2-(4-(3-(3-Chloro-4-isopropoxyphenyl)-1,2,4-oxadiazol-5-yl)phenyl)propan-2-ylamino)propanoic acid solids (289 mg) were suspended in 2-propanol/water (90/10, v/v, 2.0 mL). L-malic acid (0.7 mL, 1M, aqueous) was added and crystallization was observed. Additional 2-propanol (2.0 mL) was added. The resulting solids were filtered and analyzed.

Powder X-ray diffraction pattern and peak listing is shown in FIG. 15.

### Example 16

### (1R,3S)-3-(4-(5-(5-chloro-6-isopropoxypyridin-3-yl)-1,2,4-oxadiazol-3-yl)phenylamino)cyclopentanecarboxylic acid methanol solvate

(1R,3S)-3-(4-(5-(5-Chloro-6-isopropoxypyridin-3-yl)-1,2,4-oxadiazol-3-yl)phenylamino)cyclopentanecarboxylic acid solids (29 mg) were suspended in methanol (1.0 mL) at about 25° C. The suspension was magnetically stirred for about 3 days. The resulting solids were filtered and analyzed.

Powder X-ray diffraction pattern and peak listing is shown in FIG. 16.

### Example 17

### (1R,3S)-3-(4-(5-(5-chloro-6-isopropoxypyridin-3-yl)-1,2,4-oxadiazol-3-yl)phenylamino)cyclopentanecarboxylic acid ethanol solvate

(1R,3S)-3-(4-(5-(5-Chloro-6-isopropoxypyridin-3-yl)-1,2,4-oxadiazol-3-yl)phenylamino)cyclopentanecarboxylic acid solids (25 mg) were suspended in ethanol (1.0 mL) at about 25° C. The suspension was magnetically stirred for about 3 days. The resulting solids were filtered and analyzed.

Powder X-ray diffraction pattern and peak listing is shown in FIG. 17.

### Example 18

### (1R,3S)-3-(4-(5-(5-chloro-6-isopropoxypyridin-3-yl)-1,2,4-oxadiazol-3-yl)phenylamino)cyclopentanecarboxylic acid 2-propanol solvate

(1R,3S)-3-(4-(5-(5-Chloro-6-isopropoxypyridin-3-yl)-1,2,4-oxadiazol-3-yl)phenylamino)cyclopentanecarboxylic acid solids (28 mg) were suspended in 2-propanol (1.0 mL) at about 25° C. The suspension was magnetically stirred for about 3 days. The resulting solids were filtered and analyzed.

Powder X-ray diffraction pattern and peak listing is shown in FIG. 18.

### Example 19

### (1R,3S)-3-(4-(5-(5-chloro-6-isopropoxypyridin-3-yl)-1,2,4-oxadiazol-3-yl)phenylamino)cyclopentanecarboxylic acid 1-propanol solvate

(1R,3S)-3-(4-(5-(5-Chloro-6-isopropoxypyridin-3-yl)-1,2,4-oxadiazol-3-yl)phenylamino)cyclopentanecarboxylic acid solids (28 mg) were suspended in 1-propanol (1.0 mL) at about 25° C. The suspension was magnetically stirred for about 3 days. The resulting solids were filtered and analyzed.

Powder X-ray diffraction pattern and peak listing is shown in FIG. 19.

### Example 20

### (1R,3S)-3-(4-(5-(5-chloro-6-isopropoxypyridin-3-yl)-1,2,4-oxadiazol-3-yl)phenylamino)cyclopentanecarboxylic acid sodium salt

(1R,3S)-3-(4-(5-(5-Chloro-6-isopropoxypyridin-3-yl)-1,2,4-oxadiazol-3-yl)phenylamino)cyclopentanecarboxylic acid solids (105 mg) were suspended in ethanol/water (90/10, v/v) at 60° C. Sodium hydroxide (15 μL, 50% w/w, aqueous) was added and all solids were dissolved. The solution was removed from heat and allowed to cool to about 25° C. Crystallization was observed upon cooling. The resulting solids were filtered and analyzed.

Powder X-ray diffraction pattern and peak listing is shown in FIG. 20.

### Example 21

### (1R,3S)-3-(4-(5-(5-chloro-6-isopropoxypyridin-3-yl)-1,2,4-oxadiazol-3-yl)phenylamino)cyclopentanecarboxylic acid potassium salt

(1R,3S)-3-(4-(5-(5-Chloro-6-isopropoxypyridin-3-yl)-1,2,4-oxadiazol-3-yl)phenylamino)cyclopentanecarboxylic acid solids (32 mg) and were suspended in 1 M potassium hydroxide methanol solution (0.75 mL) for about 5 days. The resulting solids were filtered and analyzed.

Powder X-ray diffraction pattern and peak listing is shown in FIG. 21.

### Example 22

### (1R,3S)-3-(4-(5-(5-chloro-6-isopropoxypyridin-3-yl)-1,2,4-oxadiazol-3-yl)phenylamino)cyclopentanecarboxylic acid nicotinamide co-crystal

### Example 22A

(1R,3S)-3-(4-(5-(5-Chloro-6-isopropoxypyridin-3-yl)-1,2,4-oxadiazol-3-yl)phenylamino)cyclopentanecarboxylic acid solids (23 mg) were suspended in a nicotinamide solution in methanol (65 mg in 0.5 mL). The sample was sonicated for about 15 minutes. The resulting solids were filtered and analyzed.

### Example 22B

(1R,3S)-3-(4-(5-(5-Chloro-6-isopropoxypyridin-3-yl)-1,2,4-oxadiazol-3-yl)phenylamino)cyclopentanecarboxylic acid solids (180 mg) were dissolved in methanol (2.5 mL) at about 50° C. Nicotinamide solution in methanol (114 mg in 2.5 mL) was added to the solution. Crystallization was observed upon addition. The resulting solids were filtered and analyzed.

Powder X-ray diffraction pattern and peak listing is shown in FIG. 22.

### Powder X-Ray Diffractometry (PXRD)

PXRD data were collected using a G3000 diffractometer (Inel Corp., Artenay, France) equipped with a curved position sensitive detector and parallel beam optics. The diffractometer was operated with a copper anode tube (1.5 kW fine focus) at 40 kV and 30 mA. An incident beam germanium monochrometer provided monochromatic radiation Cu-K_{α} radiation, which has a wavelength of 1.5418 Å. The diffractometer was calibrated using the attenuated direct beam at one degree intervals. Calibration was checked using a silicon powder line position reference standard (NIST 640c). The instrument was computer controlled using the Symphonix software (Inel Corp., Artenay, France) and the data was analyzed using the Jade software (version 6.5, Materials Data, Inc., Livermore, Calif.). The sample was loaded onto an aluminum sample holder and leveled with a glass slide.

Characteristic powder X-ray diffraction pattern peak positions are reported for crystal forms in terms of the angular positions (two theta) with an allowable variability of ±0.2°. The variability of ±0.2° is intended to be used when comparing two powder X-ray diffraction patterns. In practice, if a diffraction pattern peak from one pattern is assigned a range of angular positions (two theta) which is the measured peak position ±0.2° and if those ranges of peak positions overlap, then the two peaks are considered to have the same angular position (two theta). For example, if a diffraction pattern peak from one pattern is determined to have a peak position of 5.2°, for comparison purposes the allowable variability allows the peak to be assigned a position in the range of 5.0°-5.4°. If a comparison peak from the other diffraction pattern is determined to have a peak position of 5.3°, for comparison purposes the allowable variability allows the peak to be assigned a position in the range of 5.1°-5.5°. Because there is overlap between the two ranges of peak positions (i.e., 5.0°-5.4° and 5.1°-5.5°) the two peaks being compared are considered to have the same angular position (two theta).

What is claimed:

1. A compound having the systematic name 3-{3-chloro-4-[5-(5-chloro-6-isopropoxy-pyridin-3-yl)-[1,2,4]oxadiazol-3-yl]phenoxy}-cyclobutane carboxylic acid (Compound 1) in a salt or crystalline form.

2. The compound of claim 1, wherein the crystalline form is free acid anhydrate, characterized by a powder X-ray diffraction pattern having at least one peak selected from those at 6.4, 7.6, 9.8, 14.1, 14.7, 16.7, 17.4, 18.5, 19.2, 19.7, 20.7, 21.3, 22.7, 24.3, 24.9, 26.1 or 26.5, each peak being ±0.2 degrees 2θ, when measured at about 25° C. with Cu K_{α} radiation at 1.5418 Å.

3. The compound of claim 1, wherein the salt form is TRIS salt, characterized by a powder X-ray diffraction pattern having at least one peak selected from those at 3.4, 6.8, 10.2, 11.7, 13.4, 15.7, 18.3, 18.9, 21.6, 21.8 22.2 or 25.0, each peak being ±0.2 degrees 2θ, when measured at about 25° C. with Cu K_{α} radiation at 1.5418 Å.

4. The compound of claim 1, wherein the crystalline form is sodium salt hydrate, characterized by a powder X-ray diffraction pattern having at least one peak selected from those at 3.2, 6.4, 9.6, 12.7, 16.0, 16.6, 17.0, 19.2, 22.5, 24.3, 25.2 or 26.5, each peak being ±0.2 degrees 2θ, when measured at about 25° C. with Cu K_{α} radiation at 1.5418 Å.

5. The compound of claim 1, wherein the salt form is potassium salt, characterized by a powder X-ray diffraction pattern having at least one peak selected from those at 4.2, 6.3, 10.6, 16.0, 16.2, 19.0, 19.4, 19.6, 20.3 or 23.3, each peak being ±0.2 degrees 2θ, when measured at about 25° C. with Cu K_{α} radiation at 1.5418 Å.

6. The compound of claim 1, wherein the crystalline form is magnesium salt, characterized by a powder X-ray diffraction pattern having at least one peak selected from those at 3.5, 7.0, 10.6, 11.5, 12.3, 13.3, 14.1, 17.0, 17.7 or 18.4, each peak being ±0.2 degrees 2θ, when measured at about 25° C. with Cu K_{α} radiation at 1.5418 Å.

7. The compound of claim 1, wherein the crystalline form is meglumine salt, characterized by a powder X-ray diffraction pattern having at least one peak selected from those at 3.1, 6.1, 9.0, 11.2, 12.7, 14.7, 15.2, 17.0, 17.8, 18.0, 19.5, 20.3 or 20.7, each peak being ±0.2 degrees 2θ, when measured at about 25° C. with Cu K_{α} radiation at 1.5418 Å.

8. The compound of claim 1, wherein the compound is a free acid anhydrate and the lattice type is triclinic, the space group is P-1, a is about 6.500 Å, b is about 11.814 Å, c is about 13.903 Å, α is about 88.21°, β is about 77.20°, γ is about 78.23°, and Z is two.

9. The compound of claim 1 wherein the compound is the sodium salt hydrate and the lattice type is triclinic, the space group is P-1, a is about 5.490 Å, b is about 8.441 Å, c is about 27.342 Å, α is about 88.84°, β is 88.20°, and γ is about 76.19°, and Z is two.

10. A pharmaceutical composition comprising a compound of claim 1 and one or more pharmaceutically acceptable excipients.

11. A pharmaceutical composition comprising a compound of claim 2 and one or more pharmaceutically acceptable excipients.

12. A pharmaceutical composition comprising a compound of claim 3 and one or more pharmaceutically acceptable excipients.

13. A pharmaceutical composition comprising a compound of claim 4 and one or more pharmaceutically acceptable excipients.

14. A pharmaceutical composition comprising a compound of claim 5 and one or more pharmaceutically acceptable excipients.

15. A pharmaceutical composition comprising a compound of claim 6 and one or more pharmaceutically acceptable excipients.

16. A pharmaceutical composition comprising a compound of claim 7 and one or more pharmaceutically acceptable excipients.

17. A pharmaceutical composition comprising a compound of claim 8 and one or more pharmaceutically acceptable excipients.

18. A pharmaceutical composition comprising a compound of claim 9 and one or more pharmaceutically acceptable excipients.

19. A compound having the systematic name 3-(2-(4-(3-(3-chloro-4-isopropoxyphenyl)-1,2,4-oxadiazol-5-yl)phenyl)propan-2-ylamino)propanoic acid (Compound 2) in a salt, solvate or crystalline form.

20. The compound of claim 19, wherein the crystalline form is methanol solvate, characterized by a powder X-ray diffraction pattern having at least one peak selected from those at 5.9, 6.8, 10.8, 11.9, 12.5, 13.6, 16.6, 17.1, 17.8, 18.6, 21.8, 24.0 or 27.81, each peak being ±0.2 degrees 2θ, when measured at about 25° C. with Cu K_{α} radiation at 1.5418 Å.

21. The compound of claim 19, wherein the crystalline form is ethanol solvate, characterized by a powder X-ray diffraction pattern having at least one peak selected from those at 5.9, 6.7, 10.7, 12.5, 13.4, 16.5, 16.8, 17.7, 20.1, 21.6, 23.8 or 27.5 each peak being ±0.2 degrees 2θ, when measured at about 25° C. with Cu K_{α} radiation at 1.5418 Å.

22. The compound of claim 19, wherein the crystalline form is 2-propanol solvate, characterized by a powder X-ray diffraction pattern having at least one peak selected from those at 5.0, 6.0, 9.6, 11.0, 15.5, 17.0, 18.0, 20.8, 21.9, 22.6, 24.1 or 25.4 each peak being ±0.2 degrees 2θ, when measured at about 25° C. with Cu K_{α} radiation at 1.5418 Å.

23. The compound of claim 19, wherein the crystalline form is 1-propanol solvate, characterized by a powder X-ray diffraction pattern having at least one peak selected from those at 6.9, 8.2, 9.9, 11.0, 11.4, 13.8, 15.0, 16.0, 16.3, 17.2, 18.6, 20.0, 23.1, 23.9, 24.5 or 25.3, each peak being ±0.2 degrees 2θ, when measured at about 25° C. with Cu K_{α} radiation at 1.5418 Å.

24. The compound of claim 19, wherein the crystalline form is ethyl acetate solvate, characterized by a powder X-ray diffraction pattern having at least one peak selected from those at 3.3, 6.6, 9.8, 11.2, 13.4, 14.5, 15.5, 16.4, 20.3, 23.0 or 24.5, each peak being ±0.2 degrees 2θ, when measured at about 25° C. with Cu K_{α} radiation at 1.5418 Å.

25. The compound of claim 19, wherein the crystalline form is toluene solvate, characterized by a powder X-ray diffraction pattern having at least one peak selected from those at 3.3, 6.6, 9.9, 13.1, 13.4, 13.7, 15.5, 16.4, 17.3, 20.4, 23.1 or 24.6, each peak being ±0.2 degrees 2θ, when measured at about 25° C. with Cu K_{α} radiation at 1.5418 Å.

26. The compound of claim 19, wherein the salt form is hydrochloride salt, characterized by a powder X-ray diffraction pattern having at least one peak selected from those at 6.1, 9.2, 12.1, 12.9, 15.9, 17.3, 17.5, 17.8, 18.2, 20.5, 22.1, 23.1, 24.4, 24.8, 25.4, 27.5 or 28.2, each peak being ±0.2 degrees 2θ, when measured at about 25° C. with Cu K_{α} radiation at 1.5418 Å.

27. The compound of claim 19, wherein the salt form is bitartrate salt, characterized by a powder X-ray diffraction pattern having at least one peak selected from those at 4.0, 10.1, 11.2, 11.6, 14.1, 14.7, 15.7, 17.1, 18.5, 18.9, 19.6, 23.6, 24.1, 24.4, 25.0, 25.4, 25.7, 26.5 or 27.0, each peak being ±0.2 degrees 2θ, when measured at about 25° C. with Cu K_{α} radiation at 1.5418 Å.

28. The compound of claim 19, wherein the salt form is bimalate salt, characterized by a powder X-ray diffraction pattern having at least one peak selected from those at 10.2, 11.3, 11.8, 14.3, 15.7, 16.6, 18.9, 19.9, 23.6, 24.2, 24.5, 24.9, 26.0 or 26.9, each peak being ±0.2 degrees 2θ, when measured at about 25° C. with Cu K_{α} radiation at 1.5418 Å.

29. A pharmaceutical composition comprising a compound of claim 19 and one or more pharmaceutically acceptable excipients.

30. A pharmaceutical composition comprising a compound of claim 20 and one or more pharmaceutically acceptable excipients.

31. A pharmaceutical composition comprising a compound of claim 21 and one or more pharmaceutically acceptable excipients.

32. A pharmaceutical composition comprising a compound of claim 22 and one or more pharmaceutically acceptable excipients.

33. A pharmaceutical composition comprising a compound of claim 23 and one or more pharmaceutically acceptable excipients.

34. A pharmaceutical composition comprising a compound of claim 24 and one or more pharmaceutically acceptable excipients.

35. A pharmaceutical composition comprising a compound of claim 25 and one or more pharmaceutically acceptable excipients.

36. A pharmaceutical composition comprising a compound of claim 26 and one or more pharmaceutically acceptable excipients.

37. A pharmaceutical composition comprising a compound of claim 27 and one or more pharmaceutically acceptable excipients.

38. A pharmaceutical composition comprising a compound of claim 28 and one or more pharmaceutically acceptable excipients.

39. A compound having the systematic name (1R,3S)-3-(4-(5-(5-chloro-6-isopropoxypyridin-3-yl)-1,2,4-oxadiazol-3-yl)phenylamino)cyclopentanecarboxylic acid (Compound 3) in a salt, solvate or crystalline form.

40. The compound of claim 39, wherein the crystalline form is methanol solvate, characterized by a powder X-ray diffraction pattern having at least one peak selected from those at 4.7, 5.8, 6.6, 7.0, 9.9, 10.3, 13.6, 13.9, 14.3, 14.7, 15.9, 16.4, 17.3, 17.5, or 20.0 each peak being ±0.2 degrees 2θ, when measured at about 25° C. with Cu K_{α} radiation at 1.5418 Å.

41. The compound of claim 39, wherein the crystalline form is ethanol solvate, characterized by a powder X-ray diffraction pattern having at least one peak selected from those at 6.3, 7.4, 11.6, 12.5, 14.7, 15.6, 17.1, 18.7, 19.1, 20.2, 23.1, 23.3, 24.5 or 25.7 each peak being ±0.2 degrees 2θ, when measured at about 25° C. with Cu K_{α} radiation at 1.5418 Å.

42. The compound of claim 39, wherein the crystalline form is 2-propanol solvate, characterized by a powder X-ray diffraction pattern having at least one peak selected from those at 5.6, 6.9, 9.3, 13.8, 15.4, 16.8, 18.7, 19.7, 22.0, 22.8 or 25.3 each peak being ±0.2 degrees 2θ, when measured at about 25° C. with Cu K_{α} radiation at 1.5418 Å.

42. The compound of claim 39, wherein the crystalline form is 1-propanol solvate, characterized by a powder X-ray diffraction pattern having at least one peak selected from those at 5.6, 6.8, 8.8, 11.6, 13.1, 13.6, 15.1, 17.6, 18.1 or 20.4 each peak being ±0.2 degrees 2θ, when measured at about 25° C. with Cu K_{α} radiation at 1.5418 Å.

43. The compound of claim 39, wherein the salt form is sodium salt, characterized by a powder X-ray diffraction pattern having at least one peak selected from those at 2.8, 5.6, 8.4, 11.2, 14.0, 15.3, 16.8, 18.1, 18.7, 19.8, 20.5, 24.9 or 26.5 each peak being ±0.2 degrees 2θ, when measured at about 25° C. with Cu K_{α} radiation at 1.5418 Å.

44. The compound of claim 39, wherein the salt form is potassium salt, characterized by a powder X-ray diffraction pattern having at least one peak selected from those at 4.5, 4.7, 8.7, 10.1, 10.9, 12.4, 12.9, 13.5, 13.9, 14.1, 16.1, 16.7, 17.1, 17.5, 17.9, 21.9, 23.5, 24.0 or 24.5 each peak being ±0.2 degrees 2θ, when measured at about 25° C. with Cu K_{α} radiation at 1.5418 Å.

45. The compound of claim 39, wherein the crystal form is nicotinamide co-crystal, characterized by a powder X-ray diffraction pattern having at least one peak selected from those at 6.4, 7.3, 11.4, 11.8, 14.8, 17.5, 22.3, 25.4, 25.9, or 27.3 each peak being ±0.2 degrees 2θ, when measured at about 25° C. with Cu K_{α} radiation at 1.5418 Å.

46. A pharmaceutical composition comprising a compound of claim 39 and one or more pharmaceutically acceptable excipients.

47. A pharmaceutical composition comprising a compound of claim 40 and one or more pharmaceutically acceptable excipients.

48. A pharmaceutical composition comprising a compound of claim 41 and one or more pharmaceutically acceptable excipients.

49. A pharmaceutical composition comprising a compound of claim 42 and one or more pharmaceutically acceptable excipients.

50. A pharmaceutical composition comprising a compound of claim 43 and one or more pharmaceutically acceptable excipients.

51. A pharmaceutical composition comprising a compound of claim 44 and one or more pharmaceutically acceptable excipients.

52. A pharmaceutical composition comprising a compound of claim 45 and one or more pharmaceutically acceptable excipients.
